(19) United States
(12) Patent Application Publication
(10) Pub. No.: US 2024/0197864 A1
(43) Pub. Date: Jun. 20, 2024

(54) ORAL COMPOSITIONS AND USE OF SAME IN VACCINATION

(71) Applicant: MIGVAX LTD., Kiryat Shmona (IL)

(72) Inventors: Sigal KREMER-TAL, Ramat-Hasharon (IL); Morris LASTER, Jerusalem (IL); Jacob PITCOVSKI, Korazim (IL)

(73) Assignee: MIGVAX LTD., Kiryat Shmona (IL)

(21) Appl. No.: 18/278,682

(22) PCT Filed: Feb. 24, 2022
(86) PCT No.: PCT/IL2022/050217

§ 371 Date: Aug. 24, 2023

Related U.S. Application Data
(60) Provisional application No. 63/153,545, filed on Feb. 25, 2021.
(60) Provisional application No. 63/184,942, filed on May 6, 2021.

(51) Int. Cl. A61K 39/295; A61K 39/00; A61K 39/108; A61K 39/215; A61P 31/14

(52) U.S. Cl. CPC A61K 39/295; A61K 39/0258; A61K 39/215; A61P 31/14; A61K 2039/53; A61K 2039/542; A61K 2039/55566; A61K 2039/6037; A61K 2039/645; C12N 2770/20034

(57) ABSTRACT

The present invention is directed to a method for increasing immunization efficiency of a subject to a pathogen, including administering a therapeutically effective amount of a pharmaceutical composition including: (a) a heat labile toxin subunit B (LTB) polypeptide or a functional analog thereof, and (b) an immunogenic polypeptide derived from the pathogen.

### CROSS-REFERENCE TO RELATED APPLICATIONS

This application claims the benefit of priority of U.S. Provisional Patent Application No. 63/153,545, titled “ORAL COMPOSITIONS AND USE OF SAME IN VACCINATION”, filed Feb. 25, 2021, and of U.S. Provisional Patent Application No. 63/184,942, titled “ORAL COMPOSITIONS AND USE OF SAME IN VACCINATION”, filed May 6, 2021. The contents of both applications are incorporated herein by reference in their entirety.

### FIELD OF THE INVENTION

The present invention relates in general to the field of vaccines.

### BACKGROUND

Vaccines are delivered through various administration routes, including parenteral routes like intramuscular or subcutaneous injection, and mucosal routes through intranasal, oral, vaginal, or rectal tract. The benefits of mucosal vaccination include: 1) strong mucosal immunity besides systemic immune responses, which provides the first barrier against those infections initiating at the mucosal surface; 2) better patient compliance due to needle free administration; 3) potential to overcome the barrier of the pre-existing immunity caused by previous parenteral vaccinations.

Among different mucosal routs, oral delivery is advantageous considering its superior patient compliance, easy administration, and mass immunization capacity, especially when it comes to the plant-derived protein antigens and veterinary vaccines.

Live viral vectors are widely used as delivery systems in mucosal vaccination, including adenovirus, attenuated influenza virus, Venezuelan equine virus, and poxvirus vectors. Besides viral vectors, nucleic acid-based vaccines such as plasmid DNA and RNA, are also being developed. The limitations of these vaccination strategies include difficulties in microorganism culturing and some safety concerns such as the possibility of reverting to the virulent state in immunocompromised hosts, as well as potential adverse effects including allergic and autoimmune reactions. Furthermore, the introduction of foreign DNA into the body could affect a cell's normal protein expression pathways. In contrast, vaccines with protein antigens are intrinsically safer than the whole pathogen-based and DNA-based antigens due to the absence of genetic materials.

Protein antigens are widely exploited in vaccine development to protect against infectious diseases. Currently there have not been any approved oral or intranasal protein vaccines yet, but extensive efforts have been reported on mucosal vaccination with protein-based antigens against various infectious diseases such as influenza, tetanus, diphtheria, hepatitis, HIV, SARS-CoV and MERS-COV, but most of these protein-based antigens are seriously limited by the generally low stability and immunogenicity to induce concerted humoral and cellular immune responses.

The usage of live bacterial cells as vehicles to deliver recombinant antigens may overcome some of these limitations by activation of the innate immune responses, thus acting as useful immunostimulating adjuvants. Many bacterial species such as attenuated strains of Salmonella enterica, Listeria monocytogenes, Streptococcus gordonii, Vibrio cholerae, Mycobacterium bovis (BCG), Yersinia enterocolitica, Shigella flexnery, as well as different Lactic acid bacteria, have been reported as promising candidates for recombinant protein delivery.

LTB is a non-toxic subunit of LT toxin expressed by enterotoxigenic E. coli strains (ETEC), responsible for binding to the host GM1 ganglioside receptors. Moreover, LTB is known to be strong mucosal adjuvant through T cells activation, although the mechanism by which it acts remains unclear. Both these traits make LTB an attractive candidate for different mucosal vaccination strategies.

However, even in view of the extensive work invested in the development of novel vaccines, there is still an urgent need for improved vaccines, for example for use in increasing immunization efficiency of a reconvalescent subject or previously vaccinated subject.

### SUMMARY

The present invention, in some embodiments, is based, in part, on the finding that one oral administration of a pharmaceutical composition as disclosed herein comprising a viral spike protein (S1) receptor binding domain (RBD), after two injections comprising the S1, resulted with immunoglobulin A (IgA) levels at least as high as obtained with three injections comprising the S1. Therefore, it is suggested herein to orally administer the S1 RBD (as a “booster”) to a subject previously been administered by means of injections with the S1 or a subject being characterized by having above a threshold level of anti S1 antibodies, so as to improve the immunization reaction of the subject. Such oral administration clearly provides an alternative and/or improved practice of vaccination, possibly with increased compliance, e.g., in young subjects, subjects found unsuitable for vaccination comprising the S1 delivered by injection, etc.

According to a first aspect, there is provided a method for increasing immunization efficiency of a subject to a pathogen, the method comprising administering a therapeutically effective amount of a pharmaceutical composition comprising: (a) a heat labile toxin subunit B (LTB) polypeptide or a functional analog thereof; and (b) an immunogenic polypeptide derived from the pathogen, thereby, increasing immunization efficiency of a subject to the pathogen.

In some embodiments, administering is: orally administering, topically administering, or both.

In some embodiments, the LTB comprises the amino acid sequence:

(SEQ ID NO: 1)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKN.

In some embodiments, the LTB comprises an amino acid sequence having at least 70%, 80%, 90%, 95%, 97%, 99%, or 100% sequence identity or homology to SEQ ID NO: 1, or any value and range therebetween. Each possibility represents a separate embodiment of the invention.

In some embodiments, the subject is characterized by having above a predetermined threshold of immunoglobulins having specific binding affinity to the immunogenic polypeptide.

In some embodiments, the method further comprises a step preceding the administering step comprising determining the subject comprises above a predetermined threshold of the immunoglobulins having specific binding affinity to the immunogenic polypeptide.

In some embodiments, the subject is a subject who previously diagnosed as being infected with the pathogen.

In some embodiments, the subject is at risk of developing an allergic reaction to an agent suitable for increasing stability of a vaccine active compound or any vaccine excipient.

In some embodiments, the method further comprises a step preceding the administering step, comprising determining the subject is at risk of developing an allergic reaction to any one of: an agent suitable for increasing stability of a vaccine active compound, and any vaccine excipient. In some embodiments, the vaccine is a messenger RNA (mRNA)-based vaccine.

In some embodiments, the agent is Polyethylene glycol (PEG).

In some embodiments, the subject is at risk of developing an allergic reaction to PEG.

In some embodiments, the immunogenic polypeptide is derived from a viral peptide.

In some embodiments, the pathogen is a pathogenic virus.

In some embodiments, the pathogenic virus comprises a Coronavirus.

In some embodiments, the Coronavirus comprises any one of: the Wuhan human Corona 2020 (SARS-Cov2), SARS-COV, or MERS-COV, or any variant thereof.

In some embodiments, the subject has been afflicted with COVID-19, had been vaccinated against COVID-19, or both.

In some embodiments, the subject is characterized by having above a predetermined threshold of immunoglobulins having specific binding affinity to at least one peptide being derived from SARS-COV-2.

In some embodiments, increasing immunization efficiency comprises inducing production, increasing levels, or both, of systemic neutralizing antibodies, in the subject.

In some embodiments, increasing immunization efficiency comprises increasing any one of: the serum titer of IgG, mucosal IgA, mucosal antibody response, T cell immune response, or any combination thereof, in the subject.

In some embodiments, the T cell comprises any one of a cytotoxic T cell and a T helper cell.

In some embodiments, the functional analog is characterized by having at least 70%, 80%, 90%, 95%, 97%, 99%, or 100% sequence identity or homology to the LTB, or any value and range therebetween. Each possibility represents a separate embodiment of the invention.

In some embodiments, the LTB polypeptide comprises a plurality of LTB polypeptides.

In some embodiments, the immunogenic polypeptide comprises a plurality of immunogenic polypeptides comprising at least two viral peptides or any analogs thereof having at least 80% sequence identity to the at least two viral peptides.

In some embodiments, the plurality of immunogenic polypeptides comprises at least two viral peptides or any analogs thereof having at least 80% sequence identity to the at least two viral peptides.

In some embodiments, the pharmaceutical composition comprises a first viral peptide, and the LTB conjugated to at least a second viral peptide, thereby forming a chimeric polypeptide.

In some embodiments, the plurality of LTB polypeptides comprises: (i) at least a first LTB polypeptide being a non-conjugated LTB; (ii) at least a second LTB polypeptide conjugated to at least one peptide of the plurality of immunogenic polypeptides, thereby forming a chimeric polypeptide; or (iii) any combination of (i) and (ii).

In some embodiments, the plurality of LTB polypeptides comprises at least a first LTB polypeptide being a non-conjugated LTB and at least a second LTB polypeptide conjugated to at least one peptide of the plurality of immunogenic polypeptides, thereby forming a chimeric polypeptide.

In some embodiments, the at least two viral peptides comprise (a) a viral spike protein; and (b) a viral nucleocapsid protein, wherein the at least two viral peptides comprise the full length amino acid sequence or a partial amino acid sequence of the viral spike protein and of the viral nucleocapsid protein, or an analog of any one of the spike protein and of the nucleocapsid protein, having at least 80% sequence identity to any one of the spike protein and the nucleocapsid protein.

In some embodiments, the spike protein comprises the amino acid sequence:

(SEQ ID NO: 15)
ITNLCPFGEVENATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNGVGYQPY
RVVVLSFELLHAPATVCGPKKSTNL;
or
any analog thereof having at least 80% sequence identity thereto.

In some embodiments, the spike protein comprises the amino acid sequence:

(SEQ ID NO: 2)
VNLTTRTQLPPAYTNSFTRGVYYPDKVFRSSVLHSTQDLFLPFFSNVTWFHAIHVSGING
TKRFDNPVLPFNDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNFKNLREFVF
KNIDGYFKIYSKHTPINLVRDLPQGFSALEPLVDLPIGINITRFQTLLALHRSYLTPGDSSS
GWTAGAAAYYVGYLQPRTFLLKYNENGTITDAVDCALDPLSETKCTLKSFTVEKGIYQ
TSNFRVQPTESIVRFPNITNLCPFGEVENATRFASVYAWNRKRISNCVADYSVLYNSASF
STFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQ
SYGFQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGIC
ASYQTQTNSPRRAR;
or
any analog thereof having at least 80% sequence identity thereto.

In some embodiments, the nucleocapsid protein comprises the amino acid sequence: (a)

(SEQ ID NO: 3)
NNTASWFTALTQHGKEDLKFPRGQGVPINTNSSPDDQIGYYRRATRRIRGGDGKMKDL
SPRWYFYYLGTGPEAGLPYGANKDGIIWVATEGALNTPKDHIGTRNPANNAAIVLQLPQ
GTTLPKGFYAEGS;
(b)
(SEQ ID NO: 4)
AAEASKKPRQKRTATKAYNVTQAFGRRGPEQTQGNFGDQELIRQGTDYKHWPQIAQFA
PSASAFFGMSRIGMEVTPSGTWLTYTGAIKLDDKDPNFKDQVILLNKHIDAYKT;
(c)
(SEQ ID NO: 10)
MSDNGPQNQRNAPRITFGGPSDSTGSNQNGERSGARSKQRRPQGLPNNTASWFTALTQ
HGKEDLKFPRGQGVPINTNSSPDDQIGYYRRATRRIRGGDGKMKDLSPRWYFYYLGTG
PEAGLPYGANKDGIIWVATEGALNTPKDHIGTRNPANNAAIVLQLPQGTTLPKGFYAEG
SRGGSQASSRSSSRSRNSSRNSTPGSSRGTSPARMAGNGGDAALALLLLDRLNQLESKM
SGKGQQQQGQTVTKKSAAEASKKPRQKRTATKAYNVTQAFGRRGPEQTQGNFGDQEL
IRQGTDYKHWPQIAQFAPSASAFFGMSRIGMEVTPSGTWLTYTGAIKLDDKDPNFKDQV
ILLNKHIDAYKTFPPTEPKKDKKKKADETQALPQRQKKQQTVILLPAADLDDFSKQLQQ
SMSSADSTQA;
or
(d)
any analog having at least 80% sequence identity to SEQ ID NO: 3,
SEQ ID NO: 4, or SEQ ID NO: 10.

In some embodiments, the conjugated is via a peptide linker comprising an amino acid sequence of 2 to 10 amino acids.

In some embodiments, the linker comprises 3 to 7 amino acids.

In some embodiments, the linker comprises Serine and Glycine amino acid residues.

In some embodiments, the linker consists of Serine and Glycine amino acid residues.

In some embodiments, the pharmaceutical composition comprises: (a) an LTB polypeptide being a non-conjugated LTB; and the at least two viral peptides; (b) a first LTB polypeptide being a non-conjugated LTB; a first viral peptide; and a chimeric polypeptide comprising at least a second LTB polypeptide conjugated to at least a second viral peptide; (c) a first chimeric polypeptide comprising a first LTB polypeptide conjugated to at least a first viral peptide and a second chimeric polypeptide comprising at least a second LTB polypeptide conjugated to at least a second viral peptide; (d) a first viral peptide; and a chimeric polypeptide comprising an LTB polypeptide conjugated to at least a second viral peptide; or (e) any combination of (a) to (d).

In some embodiments, the composition comprises LTB, and RBD of a spike protein and/or a nucleocapsid protein.

In some embodiments, the composition comprises a chimera comprising LTB, and RBD of a spike protein. In some embodiments, the composition comprising a chimera comprising LTB, and RBD of a spike protein further comprises a nucleocapsid protein.

In some embodiments, the composition comprises a chimera comprising LTB, and a nucleocapsid protein. In some embodiments, the composition comprising a chimera comprising LTB, and a nucleocapsid protein further comprises RBD of a spike protein.

In some embodiments, the composition comprises LTB, an RBD of a spike protein, and a chimera comprising LTB, and a nucleocapsid protein.

In some embodiments, the composition comprises a chimera comprising LTB, RBD of a spike protein, and a nucleocapsid protein.

In some embodiments, the chimeric polypeptide comprises the sequence of:

(SEQ ID NO: 9)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNEFITNLCPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTNG
VGYQPYRVVVLSFELLHAPATVCGPKKSTNLVKNKXVNFNFNGLTGT,
wherein X is cysteine or alanine.

In some embodiments, the chimeric polypeptide comprises the sequence of:

(SEQ ID NO: 5)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGVNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDPLSETKCTLK
SFTVEKGIYQTSNFRVQPTESIVRFPNITNLCPFGEVENATRFASVYAWNRKRISNCVAD
YSVLYNSASFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQAGSTPCNG
VEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLIGAEHVNNS
YECDIPIGAGICASYQTQTNSPRRAR.

In some embodiments, the chimeric polypeptide comprises the sequence of:

(SEQ ID NO: 6)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGNNTASWFTALTQHGKEDLKFPRGQGVPINTNSSPDDQIGYYRRATRRIR
GGDGKMKDLSPRWYFYYLGTGPEAGLPYGANKDGIIWVATEGALNTPKDHIGTRNPAN
NAAIVLQLPQGTTLPKGFYAEGS.

In some embodiments, the chimeric polypeptide comprises the sequence of:

(SEQ ID NO: 7)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGAAEASKKPRQKRTATKAYNVTQAFGRRGPEQTQGNFGDQELIRQGTDY
KHWPQIAQFAPSASAFFGMSRIGMEVTPSGTWLTYTGAIKLDDKDPNFKDQVILLNKHI
DAYKT.

In some embodiments, the chimeric polypeptide comprises the sequence of:

(SEQ ID NO: 11)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGKMKDLSPRW
YFYYLGTGPEAGLPYGANKDGIIWVATEGALNTPKDHIGTRNPANNAAIVLQLPQGTTL
PKGFYAEGSRGGSQASSRSSSRSRNSSRNSTPGSSRGTSPARMAGNGGDAALALLLLDR
LNQLESKMSGKGQQQQGQTVTKKSAAEASKKPRQKRTATKAYNVTQAFGRRGPEQTQ
GNFGDQELIRQGTDYKHWPQIAQFAPSASAFFGMSRIGMEVTPSGTWLTYTGAIKLDDK
DPNFKDQVILLNKHIDAYKTFPPTEPKKDKKKKADETQALPQRQKKQQTVTLLPAADL
DDFSKQLQQSMSSADSTQA.

In some embodiments, the increasing is compared to a control subject.

In some embodiments, the subject had previously been vaccinated by an intramuscular injection or a subcutaneous injection of a composition comprising a SARS-COV2 spike protein, and is orally administered with a therapeutically effective amount of a pharmaceutical composition comprising a SARS-COV2 spike protein receptor binding domain as an immunogenic polypeptide.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include vertical bar graphs showing that a heterologous oral boosting using the spike protein (S1) receptor binding domain (RBD) subsequently to S1 injection(s) effectively induce the production of neutralizing antibodies. (1A) Anti-SI immunoglobulin G (IgG) in sera (1B) Pseudo-neutralization. Injection-S1 (I); oral placebo (O_{(P)}); Oral mixture of LTB, LTB-NC and LTB-NN fusion proteins and SARS-Cov-2 Spike protein RBD domain (“Migvax 101a”; O_{(M)}); and serum sampled (S). * P<0.05, *** P<0.001, no statistically significant difference (n.s.).

FIG. 2 includes a vertical bar graph showing quantification of anti-SI immunoglobulin A (IgA) in bronchoalveolar lavage fluid (BALF). Values obtained for anti-SI IgA were divided by the total IgA value for each sample (e.g., to normalize values for differences in the total IgA extraction). Injection-S1 (I); oral placebo (O_{(P)}); Oral mixture of LTB, LTB-NC and LTB-NN fusion proteins and SARS-Cov-2 Spike protein RBD domain (“Migvax 101a”; O_{(M)}); and serum sampled (S). * P<0.05, *** P<0.001, no statistically significant difference (n.s.).

FIG. 3 includes a scheme of a non-limiting study design as presented herein.

FIGS. 4A-4B include graphs showing levels of coronavirus shedding in an avian model organism. Specifically shown is IBV shedding in birds following IBV challenge. (strain M41) vaccine, or orally administered bacterial Caye broth growth medium (neg), wild type E. coli (WT), or E. coli strains secreting the LTB-fused polypeptides: LS1, 1:1 ratio of LNC and LNN (LNC+LNN) or 1:1:1 ratio of LS1, LNC and LNN (LS1+LNC+LNN). Graphs show the IBV titres shed by chickens, as determined by qRT-PCR performed on swab samples collected from either the choana (4A) or the cloaca (4B), before challenge (0), and 3, 6 and 10 days, post-infection (DPI). The differences between groups at 3, 6 and 10 DPI were analysed by one-way-ANOVA with Tukey HSD post-hoc. Statistical analyses for groups at 3, 6 and 10 DPI are presented in orange, purple and green respectively.

FIG. 5 includes a vertical bar graph showing the vaccine protein composition as disclosed herein reduces infectivity of a coronavirus in an avian model organism. Specifically presented is percentage of birds that shed IBV from either the choana or the cloaca following IBV challenge. Chickens were challenged with live virulent IBV (strain M41) 15 days after administration of a last booster dose of either a subcutaneously injected inactivated IBV (strain M41) vaccine, or orally administered bacterial Caye broth growth medium (neg), wild type E. coli (WT), or E. coli strains secreting the LTB-fused polypeptides: LS1, 1:1 ratio of LNC and LNN (LNC+LNN) or 1:1:1 ratio of LS1, LNC and LNN (LS1+LNC+LNN). Graphs show the percent of shedding chickens as determined by qRT-PCR performed on swab samples taken from either the choana or the cloaca, before challenge (0), and 3, 6 and 10 days, post-infection (DPI). The differences between groups at 10 DPI were analysed by Fisher's exact test.

### DETAILED DESCRIPTION

In some embodiments, the present invention is directed to a method for increasing, improving, enhancing, any equivalent thereof, or any combination thereof, an immunization efficiency, compliance, or both, of a subject.

In some embodiments, the present invention comprises a composition comprising LTB and an immunogenic polypeptide, suitable for increasing an immunization efficiency of a subject to a pathogen.

In some embodiments, the composition comprises LTB and at least one viral peptide, a functional fragment thereof, or an analog thereof.

In some embodiments, LTB comprises a plurality of LTB.

In some embodiments, LTB comprises a functional analog of LTB.

In some embodiments, the immunogenic polypeptide comprises a plurality of immunogenic polypeptides.

In some embodiments, the at least one viral peptide comprises a plurality of viral peptides.

In some embodiments, the composition comprises at least one chimeric polypeptide comprising at least a first LTB polypeptide and at least a first viral peptide of the plurality of immunogenic polypeptides.

In some embodiments, there is provided a chimeric polypeptide comprising LTB and at least one immunogenic polypeptide, such as a viral peptide.

In some embodiments, the plurality of immunogenic polypeptides comprise at least one viral peptide derived from a Coronavirus. As used herein, the terms “Coronavirus” or “Coronaviruses” encompass any virus belonging to the family of “Coronaviridae”. In some embodiments, the Coronavirus is selected from any of the genera: Alphacoronavirus, Betacoronavirus, Gammacoronavirus, and Deltacoronavirus.

### Methods

According to some embodiments, there is provided a method for increasing immunization efficiency of a subject to a pathogen, the method comprising administering a therapeutically effective amount of a pharmaceutical composition comprising: an immunogenic polypeptide derived from a pathogen, thereby, increasing immunization efficiency of a subject to the pathogen.

In some embodiments, administering comprises orally administering. In some embodiments, administering comprises topically administering. In some embodiments, administering comprises orally administering and topically administering.

In some embodiments, the administering comprises a single administering.

In some embodiments, the administering comprises multiple administering.

In some embodiments, multiple administering events are at least 1 week apart, at least 2 weeks apart, at least 3 weeks apart, or at least 4 weeks apart, or any value and range therebetween. Each possibility represents a separate embodiment of the invention.

In some embodiments, multiple administering events are 1-3 weeks apart, 2-3 weeks apart, 1-5 weeks apart, or 2-6 weeks apart. Each possibility represents a separate embodiment of the invention.

In some embodiments, post a first topical administration, the level of neutralizing antibodies is increased by 1-10%, 2-20%, 5-15%, or 1-20%, compared to a control. Each possibility represents a separate embodiment of the invention.

In some embodiments, post a second topical administration, the level of neutralizing antibodies is increased by 20-70%, 25-70%, 30-65%, 35-70%, 40%, 50-65%, or 60-70%, compared to a control. Each possibility represents a separate embodiment of the invention.

In some embodiments, post a third topical administration, the level of neutralizing antibodies is increased by 70-100%, 85-99%, 80-97%, 80-100%, 90-99%, or 90-100%, compared to a control. Each possibility represents a separate embodiment of the invention.

In some embodiments, a control comprises the level of neutralizing antibodies in an injected subject. In some embodiments, a control comprises the level of neutralizing antibodies in a transdermally injected subject. In some embodiments, the control subject is vaccinated at least three times by subcutaneous injection, intramuscular injection, transdermal injection, or any combination thereof. In some embodiments, the injection, e.g., intramuscular, subcutaneous, etc., comprises administration of a composition comprising a SARS-COV2 spike protein (S1) or a polynucleotide encoding same (e.g., a transcript).

According to some embodiments, the pharmaceutical composition comprises: (a) a heat labile toxin subunit B (LTB) polypeptide or a functional analog thereof; and (b) an immunogenic polypeptide derived from a pathogen, thereby, increasing immunization efficiency of a subject to the pathogen.

In some embodiments, increasing immunization comprises reducing infectivity.

In some embodiments, a subject administered with the herein disclosed composition is less infective.

In some embodiments, reducing infectivity comprises reducing shedding of a virus as disclosed herein.

As used herein, the term “viral shedding” refers to expulsion and thereafter release of viral particle resulting from successful reproduction during a host cell infection, as would be apparent to one of ordinary skill in the art.

In some embodiments, reducing comprises at least 5%, at least 15%, at least 25%, at least 50%, at least 75%, at least 95%, at least 97%, at least 99%, or 100% reduction, or any value and range therebetween. Each possibility represents a separate embodiment of the invention.

In some embodiments, reducing comprises 5-50%, 10-85%, 20-100%, 30-95%, 1-40%, or 15-99% reduction. Each possibility represents a separate embodiment of the invention.

In some embodiments, the LTB comprises the amino acid sequence:

(SEQ ID NO: 1)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKN.

In some embodiments, the subject is characterized by having above a predetermined threshold of immunoglobulins having specific binding affinity to the immunogenic polypeptide.

In some embodiments, the method further comprising a step preceding the administering step, comprising determining the subject comprises above a predetermined threshold of the immunoglobulins having specific binding affinity to the immunogenic polypeptide.

In some embodiments, a subject determined to comprise or characterized by having above a predetermined threshold of the immunoglobulins having specific binding affinity to the immunogenic polypeptide is suitable for treatment according to the herein disclosed method.

In some embodiments, the determining is in a sample derived or obtained from the subject. In some embodiments, the determining is in vitro determining. In some embodiments, in vitro is in a plate or a tube, or any means suitable for determining which would be apparent to one of ordinary skill in the art.

In some embodiments, the increasing is at least 5%, at least 10%, at least 25%, at least 50%, at least 100%, at least 200%, at least 350%, at least 400%, at least 500%, at least 650%, at least 750%, at least 900%, or at least 1,000%, increase, or any value and range therebetween. Each possibility represents a separate embodiment of the invention. In some embodiments, the increasing is 5-250%, 10-500%, 50-650%, 100-800%, 250-900%, or 150-1,000% increase. Each possibility represents a separate embodiment of the invention.

In some embodiments, the increasing is compared to a control subject. In some embodiments, the control subject has or had been infected by the pathogen. In some embodiments, the control subject has or had been vaccinated for or against the pathogen. In some embodiments, the control subject has or had been vaccinated for or against the pathogen, or recovered therefrom, at least 2 weeks, at least 4 weeks, at least 2 months, at least 4 months, at least 6 months, or at least 12 months before being compared to a subject treated according to the disclosed treatment. In some embodiments, the control subject has or had been vaccinated once or twice for or against the pathogen.

In some embodiments, the control subject is the same subject being treated according to the method disclosed herein, prior the disclosed treatment. In some embodiments, the control subject is the same subject being treated according to the method disclosed herein, at least 2 weeks, at least 4 weeks, at least 2 months, at least 4 months, at least 6 months, or at least 12 months, prior the disclosed treatment.

In some embodiments, the subject comprises a subject who previously diagnosed as being infected with the pathogen.

In some embodiments, the subject is at risk of developing an allergic reaction to an agent of a vaccine composition. In some embodiments, the subject is afflicted with an allergic reaction to an agent of a vaccine composition. In some embodiments, the subject had been afflicted with an allergic reaction to an agent of a vaccine composition. In some embodiments, the subject has predisposition to developing an allergic reaction to the agent.

In some embodiments, the agent is not the active agent of the vaccine.

In some embodiments, the agent is a carrier, diluent, stabilizer, adjuvant, or any combination thereof. In some embodiments, the agent is suitable for increasing stability of a vaccine active compound.

In some embodiments, the method further comprises a step comprising determining the subject is at risk of developing an allergic reaction to an agent suitable for increasing stability of a vaccine active compound.

In some embodiments, the step of determining that the subject is at risk of developing an allergic reaction to an agent suitable for increasing stability of a vaccine active compound, precedes the administering step.

In some embodiments, the vaccine comprises a messenger RNA (mRNA)-based vaccine.

As used herein, the term “mRNA-based vaccine” refers to any vaccine which utilises a messenger RNA (mRNA) encoding the vaccination antigen of choice, which is injected into a patient and is taken up by local somatic and immune cells. Once inside the cytosol the mRNA is translated and the vaccination antigen is produced as a protein or a peptide, which induces an immune response. The magnitude, duration and character of the immune response depend on the immunostimulatory context, in which the antigen is presented.

In some embodiments, the agent is or comprises polyethylene glycol (PEG).

In some embodiments, the subject is at risk of developing an allergic reaction to PEG.

In some embodiments, the immunogenic polypeptide is derived from a viral peptide.

In some embodiments, the pathogen is a pathogenic virus.

In some embodiments, the pathogenic virus comprises a Coronavirus.

In some embodiments, the Coronavirus comprises any one of: the Wuhan human Corona 2020 (SARS-Cov2), SARS-COV, or MERS-COV, including any variant or a pathogenic variant thereof.

In some embodiments, the subject is infected with or at increased risk of infection of: Coronavirus, SARS-COV, SARS-COV-2, or MERS-COV. In some embodiments, the subject is a mammal. In some embodiments, the subject is a human subject.

In some embodiments, the subject has been afflicted with COVID-19, had been vaccinated against COVID-19, or both.

In some embodiments, the subject is characterized by having above a predetermined threshold of immunoglobulins having specific binding affinity to at least one peptide being derived from SARS-COV-2.

In some embodiments, increasing immunization efficiency comprises inducing production, increasing levels, or both, of systemic neutralizing antibodies, in the subject.

In some embodiments, increasing immunization efficiency comprises increasing the titer of IgG, IgA, or both, in the subject. In some embodiments, the increased titer is in the serum, saliva, or both, of the subject.

In some embodiments, there is provided a method for heterologous boosting of a vaccination in a subject previously been afflicted with or vaccinated against COVID-19.

In some embodiments, the subject had previously been vaccinated by subcutaneous, intramuscular, transdermal, dermal, nasal, or oral, injections/administration, or any combination thereof. In some embodiments, the administration, e.g., intramuscular, subcutaneous, etc., comprises administration of a composition comprising a SARS-COV2 spike protein (S1), including, but not limited to a polynucleotide encoding same (e.g., a transcript). In some embodiments, the administration comprises administering a composition comprising a SARS-CoV2 receptor binding domain (RBD) of the S1, including, but not limited to a polynucleotide encoding same (e.g., a transcript). In some embodiments, the method comprises orally administering a therapeutically effective amount of a pharmaceutical composition comprising a SARS-COV2 spike protein receptor binding domain (RBD) as an immunogenic polypeptide, to a subject previously been vaccinated. In some embodiments, the method comprises orally administering a therapeutically effective amount of a pharmaceutical composition comprising a SARS-COV2 S1 RBD as an immunogenic polypeptide, to a subject previously been vaccinated such as by subcutaneous, intramuscular, transdermal, dermal, nasal, or oral, injections/administration, or any combination thereof, of a composition comprising or consisting essentially of S1 as an immunogenic peptide, including, but not limited to a polynucleotide encoding same.

In some embodiments, the method comprises topically administering a therapeutically effective amount of a pharmaceutical composition comprising a SARS-COV2 spike protein receptor binding domain (RBD) as an immunogenic polypeptide, to a subject previously been vaccinated. In some embodiments, the method comprises topically administering a therapeutically effective amount of a pharmaceutical composition comprising a SARS-COV2 S1 RBD as an immunogenic polypeptide, to a subject previously been vaccinated by subcutaneous injection, intramuscular injection, transdermal injection, or any combination thereof, of a composition comprising or consisting essentially of S1 as an immunogenic peptide, or a polynucleotide encoding same.

In some embodiments, the method comprises topically and orally administering a therapeutically effective amount of a pharmaceutical composition comprising a SARS-COV2 spike protein receptor binding domain (RBD) as an immunogenic polypeptide, to a subject previously been vaccinated. In some embodiments, the method comprises topically and orally administering a therapeutically effective amount of a pharmaceutical composition comprising a SARS-COV2 S1 RBD as an immunogenic polypeptide, to a subject previously been vaccinated by subcutaneous injection, intramuscular injection, transdermal injection, or any combination thereof, of a composition comprising or consisting essentially of S1 as an immunogenic peptide, or a polynucleotide encoding same.

As used herein, the term “consisting essentially of” denotes that a given compound or substance constitutes the vast majority of the active ingredient's portion or fraction of the composition.

In some embodiments, the pathogenic virus comprises an influenza virus.

In some embodiments, the at least one immunogenic antigen of Influenza virus is hemagglutinin (HA).

In some embodiments, HA comprises the amino acid sequence:

(SEQ ID NO: 18)
MKTIIALSYIFCLVFAQDLPGNDNNSTATLCLGHHAVPNGTLVKTITNDQIEVTNATELV
QSSSTGKICNNPHRILDGINCTLIDALLGDPHCDGFQNEKWDLFVERSKAFSNCYPYDVP
DYASLRSLVASSGTLEFINEGFNWTGVTQNGGSSACKRGPDSGFFSRLNWLYKSGSTYP
VQNVTMPNNDNSDKLYIWGVHHPSTDKEQTNLYVQASGKVTVSTKRSQQTIIPNVGSR
PWVRGLSSRISIYWTIVKPGDILVINSNGNLIAPRGYFKMRTGKSSIMRSDAPIGTCSSECI
TPNGSIPNDKPFQNVNKITYGACPKYVKQNTLKLATGMRNVPEKQTRGIFGAIAGFIENG
WEGMIDGWYGFRHQNSEGTGQAADLKSTQAAIDQINGKLNRVIEKTNEKFHQIEKEFSE
VEGRIQDLEKYVEDTKIDLWSYNAELLVALENQHTIDLTDSEMNKLFEKTRRQLRENAE
DMGNGCFKIYHKCDNACIGSIRNGTYDHDVYRDEALNNRFQIKGVELKSGYKDWILWI
SFAISCFLLCVVLLGFIMWACQKGNIRCNICI,
or
any functional analog thereto, having at least 50%, at least 60%, at
least 70%, at least 80%, at least 90%, at least 95%, or at least 99%
homology or identity.

The influenza HA amino acid sequence may vary among strains and/or variants. The present invention, in some embodiments, contemplates any HA as an immunogenic peptide, as part of compositions and/or chimeras, as disclosed herein.

### Composition

According to some embodiments, there is provided a composition comprising: a heat labile toxin subunit B (LTB) polypeptide and an immunogenic polypeptide, or a plurality thereof.

In some embodiments, the composition comprises a first viral peptide, and the LTB polypeptide being conjugated to at least a second viral peptide, thereby forming a chimeric polypeptide.

In some embodiments, the plurality of immunogenic polypeptides comprises at least two viral peptides or any analogs thereof having at least 80% sequence identity to the at least two viral peptides.

According to some embodiments, there is provided a composition comprising LTB or a functional analog thereto (such as, but not limited to CTB) and at least one viral peptide selected from: spike protein 1, nucleocapsid protein, any functional fragment thereof, or any analog thereof having at least 80% homology or identity thereto.

In some embodiments, the spike protein 1 fragment or analog composites the RBD of the spike protein. In some embodiments, the LTB and the at least one viral peptide are non-conjugated to one another.

According to some embodiments, there is provided a composition comprising LTB or a functional analog thereto (such as, but not limited to CTB) and at least one influenza peptide.

According to some embodiments, there is provided a composition comprising LTB or a functional analog thereto (such as, but not limited to CTB) and influenza hemagglutinin (HA) protein, any functional fragment thereof, or any analog thereof having at least 80% homology or identity thereto.

In some embodiments, the LTB polypeptide comprises a plurality of LTB polypeptides.

In some embodiments, the composition of the invention comprises a plurality of LTB polypeptides.

In some embodiments, of a plurality of LTB polypeptides, all LTB polypeptides are non-conjugated. In some embodiments, of a plurality of LTB polypeptides, all LTB polypeptides are conjugated. In some embodiments, of a plurality of LTB polypeptides, at least one LTB polypeptide is non-conjugated, and at least one LTB polypeptide is conjugated. In some embodiments, conjugated and/or non-conjugated is to at least one viral peptide, as described herein.

As used herein, the term “plurality” comprises any integer equal to or greater than 2. In some embodiments, a plurality comprises at least 2, at least 3, at least 5, at least 7, at least 9, at least 10, at least 12, or at least 15, or any value and range therebetween. Each possibility represents a separate embodiment of the invention. In some embodiments, a plurality comprises 2 to 15, 2 to 10, 2 to 8, 2 to 4, 3 to 11, 3 to 9, 3 to 7, 4 to 15, or 4 to 8. Each possibility represents a separate embodiment of the invention.

In some embodiments, the plurality of LTB polypeptides comprises: (i) at least a first LTB polypeptide being a non-conjugated LTB; (ii) at least a second LTB polypeptide conjugated to at least one peptide of the plurality of immunogenic polypeptides, thereby forming a chimeric polypeptide of the invention; or (iii) any combination of (i) and (ii).

As used herein, the term “non-conjugated” refers to a “free” peptide or a polypeptide. In some embodiments, free peptide, or polypeptide, e.g., LTB refers to a polypeptide, e.g., LTB, that is not fused to any other peptide as described herein. In some embodiments, a free LTB is devoid of any portion or partial sequence of a viral peptide as described herein, conjugated of fused thereto. In some embodiments, a free LTB comprises any LTB, such as a wildtype LTB, a modified LTB, a tag comprising LTB (such as for purification or identification), as long as the free LTB is devoid of any portion or partial sequence of a viral peptide as described herein.

In some embodiments, a tag as used herein, is connected directly or indirectly to the LTB. In some embodiments, indirectly connected is via a linker, as described herein.

As used herein, the term “conjugated” refers to a peptide or a polypeptide, e.g., LTB, being linked to at least one different peptide, e.g., the plurality of immunogenic peptides. In some embodiments, linked comprises chemically bound. In some embodiments, a chemical bond comprises a covalent bond. In some embodiments, a chemical bond comprises a peptide bond. In some embodiments, LTB is linked to the at least one peptide of the plurality of immunogenic peptides directly. In some embodiments, LTB is linked to the at least one peptide of the plurality of immunogenic peptides indirectly, such as via a linker, as disclosed herein. In some embodiments, the linker is a flexible or a rigid linker.

In some embodiments, the plurality of LTB polypeptides comprises at least a first LTB polypeptide being a non-conjugated LTB and at least a second LTB polypeptide conjugated to at least one peptide of the plurality of immunogenic polypeptides, thereby forming a chimeric polypeptide of the invention.

In some embodiments, the immunogenic polypeptides of the plurality of immunogenic polypeptides are derived from a single pathogen species. In some embodiments, the immunogenic polypeptides of the plurality of immunogenic polypeptides are derived from multiple pathogen species.

In some embodiments, the at least two viral peptides comprise (a) a viral spike protein; and (b) a viral nucleocapsid protein.

In some embodiments, the at least two viral peptides are derived from a single virus species. In some embodiments, the at least two viral peptides are derived from multiple virus species.

In some embodiments, the spike protein and the nucleocapsid protein are derived from a single virus species. In some embodiments, the spike protein and the nucleocapsid protein are derived from multiple virus species. In some embodiments, the spike protein is derived from a first virus species and the nucleocapsid protein is derived from a second virus species.

In some embodiments, the at least two viral peptides comprise the full-length amino acid sequence or a partial amino acid sequence of the viral spike protein and of the viral nucleocapsid protein.

In some embodiments, the at least two viral peptides comprise an analog of any one of: the spike protein or a receptor binding domain (RBD) of same, and the nucleocapsid protein.

In some embodiments, the spike protein or RBD thereof comprises any viral spike protein or RBD thereof. In some embodiments, the spike protein or RBD thereof comprises any coronavirus derived spike protein or RBD thereof. In some embodiments, the spike protein or RBD thereof comprises any coronavirus derived spike protein or RBD thereof as long as the spike protein or RBD thereof is essentially structurally similar or identical to the SARS-COV-2 spike protein or RBD thereof.

In some embodiments, essentially structurally identical is determined according to the level of the root mean square distancing (RMSD). In some embodiments, any viral spike protein or RBD thereof being essentially structurally similar or identical to the SARS-COV-2 spike protein or RBD thereof is contemplated according to the herein disclosed composition, and methods of using same.

In some embodiments, the spike protein has a RMSD of 0.1 at most, 0.2 at most, 0.3 at most, 0.4 at most, 0.5 at most, 0.7 at most, 0.9 at most, 1.0 at most, 1.2 at most, 1.4 at most, 1.5 at most, 1.6 at most, 1.8 at most, 1.9 at most, 2.0 at most, 2.2 at most, 2.5 at most, or 3.5 at most, corresponding to the SARS-COV-2 spike protein, or any value and range therebetween. Each possibility represents a separate embodiment of the invention.

In some embodiments, the analog has at least 15%, at least 20%, at least 30%, at least 40%, at least 50%, at least 60%, at least 70%, at least 80%, or at least 90% sequence identity to any one of: the spike protein and the nucleocapsid protein, or any value and range therebetween. Each possibility represents a separate embodiment of the invention.

In some embodiments, the composition of the invention comprises: a first LTB polypeptide being a non-conjugated LTB; and the at least two viral peptides. In some embodiments, the composition comprises a first LTB polypeptide being a non-conjugated LTB and the at least two viral peptides unconjugated to the first LTB polypeptide.

In some embodiments, the composition comprises a first LTB polypeptide being a non-conjugated LTB; a first viral peptide; and a chimeric polypeptide comprising at least a second LTB polypeptide conjugated to at least a second viral peptide, as disclosed herein.

In some embodiments, the composition comprises a first chimeric polypeptide comprising a first LTB polypeptide conjugated to at least a first viral peptide and a second chimeric polypeptide comprising at least a second LTB polypeptide conjugated to at least a second viral peptide, as disclosed herein.

In some embodiments, the composition comprises any combination of: a first LTB polypeptide being a non-conjugated LTB; and the at least two viral peptides, a first LTB polypeptide being a non-conjugated LTB; a first viral peptide; and a chimeric polypeptide comprising at least a second LTB polypeptide conjugated to at least a second viral peptide, and a first chimeric polypeptide comprising a first LTB polypeptide conjugated to at least a first viral peptide and a second chimeric polypeptide comprising at least a second LTB polypeptide conjugated to at least a second viral peptide.

In some embodiments, the composition comprises a first LTB polypeptide being a non-conjugated LTB, a first viral peptide, being a non-conjugated or free spike protein, as described herein, and a second viral peptide, being a non-conjugated or free nucleocapsid protein, as described herein.

In some embodiments, the composition comprises a first LTB polypeptide being a non-conjugated LTB; a first viral peptide, a non-conjugated or free spike protein, as described herein; and a chimeric polypeptide comprising at least a second LTB polypeptide conjugated to at least a second viral peptide, being a nucleocapsid protein, as described herein.

In some embodiments, the composition comprises a first chimeric polypeptide comprising a first LTB polypeptide conjugated to at least a first viral peptide, being a spike protein, as described herein, and a second chimeric polypeptide comprising at least a second LTB polypeptide conjugated to at least a second viral peptide, being a nucleocapsid protein, as described herein.

In some embodiments, the at least two viral peptides comprise at least one immunogenic antigen of a pathogenic virus. In some embodiments, the at least two viral peptides comprise at least 2, at least 3, at least 4, at least 5, at least 6, at least 7, at least 8, at least 9, or at least 10 immunogenic antigens, or any value and range therebetween. Each possibility represents a separate embodiment of the invention.

In some embodiments, LTB comprises a functional analog of LTB. In some embodiments, the LTB analog is characterized by having at least 80%, at least 85%, at least 90%, at least 95%, at least 97%, at least 99%, or 100% sequence identity or homology to LTB, or any value and range therebetween. Each possibility represents a separate embodiment of the invention.

In some embodiments, the functional analog is characterized by being capable of binding to a cell membrane. In some embodiments, the cell is a host cell. In some embodiments, the cell in an epithelial cell.

In some embodiments, the composition comprises a Cholera enterotoxin subunit B (CTB) polypeptide. In some embodiments, the composition comprises an LTB polypeptide or a CTB polypeptide. In some embodiments, the composition of the invention comprises a plurality of immunogenic peptides with LTB, CTB, or a combination thereof. In some embodiments, LTB and CTB are alternatives for use in the herein disclosed composition and methods. In some embodiments, a composition comprising LTB is devoid of CTB. In some embodiments, a composition comprising CTB is devoid of LTB. In some embodiments, the composition is formulated with LTB or CTB as equivalent alternatives.

In some embodiments, there is provided a pharmaceutical composition comprising the composition of the invention and a pharmaceutically acceptable carrier.

In some embodiments, there is provided a pharmaceutical composition comprising: (a) the chimeric polypeptide of the invention; or (b) the herein disclosed cell, and a pharmaceutically acceptable carrier.

In some embodiments, the pharmaceutical composition comprises (a) a chimeric polypeptide comprising the LTB or CTB polypeptide and a viral spike peptide, (b) a chimeric polypeptide comprising the LTB or CTB polypeptide and a viral nucleocapsid protein, (c) a chimeric polypeptide comprising the LTB or CTB polypeptide, a viral spike peptide, and a viral nucleocapsid protein, or any combination of (a) to (c), and a pharmaceutically acceptable carrier.

In some embodiments, the pharmaceutical composition comprises (a) LTB or CTB, and (b) a viral spike protein. In some embodiments, the pharmaceutical composition comprises (a) LTB or CTB, and (b) an RBD of a viral spike protein. In some embodiments, the pharmaceutical composition comprises (a) LTB and (b) a viral spike protein. In some embodiments, the pharmaceutical composition comprises (a) LTB and (b) an RBD of a viral spike protein. In some embodiments, the LTB or CTB and the viral spike protein are non-conjugated. In some embodiments, the LTB or CTB and the RBD of a viral spike protein are non-conjugated. In some embodiments, there is provided a pharmaceutical composition consisting essentially of LTB and a viral spike protein, wherein LTB and the viral spike protein are non-conjugated. In some embodiments, there is provided a pharmaceutical composition consisting essentially of LTB and an RBD of a viral spike protein, wherein LTB and the RBD of the viral spike protein are non-conjugated.

In some embodiments, the pharmaceutical composition comprises a cell or a plurality of cells, comprising: (a) a chimeric polypeptide comprising the LTB or CTB polypeptide and a viral spike peptide, (b) a chimeric polypeptide comprising the LTB or CTB polypeptide and a viral nucleocapsid protein, (c) a chimeric polypeptide comprising the LTB or CTB polypeptide, a viral spike peptide, and a viral nucleocapsid protein, or any combination of (a) to (c), and a pharmaceutically acceptable carrier.

In some embodiments, the pharmaceutical composition comprises any one of: (a) LTB or CTB, and influenza HA protein; (b) LTB or CTB, and a chimeric polypeptide comprising the LTB or CTB polypeptide and a hemagglutinin (HA) protein; or a combination of (a) and (b), and a pharmaceutically acceptable carrier.

In some embodiments, the pharmaceutical composition comprises (a) LTB or CTB, and (b) a hemagglutinin (HA) protein. In some embodiments, the LTB or CTB and the hemagglutinin (HA) protein are non-conjugated. In some embodiments, the LTB or CTB and the hemagglutinin (HA) protein are conjugated. In some embodiments, there is provided a pharmaceutical composition consisting essentially of LTB or CTB and a hemagglutinin (HA) protein, wherein LTB or CTB and the hemagglutinin (HA) protein are non-conjugated. In some embodiments, there is provided a pharmaceutical composition consisting essentially of LTB or CTB and hemagglutinin (HA) protein, wherein LTB or CTB and the hemagglutinin (HA) protein are conjugated.

In some embodiments, the pharmaceutical composition comprises a cell or a plurality of cells, comprising: (a) a chimeric polypeptide comprising the LTB or CTB polypeptide and hemagglutinin (HA) protein, (b) a chimeric polypeptide comprising the LTB or CTB polypeptide and a hemagglutinin (HA) protein, or a combination of (a) and (b), and a pharmaceutically acceptable carrier.

In some embodiments, the pharmaceutical composition is formulated for an oral administration route, topical administration, or both.

The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable, or synthetic origin, such as peanut oil, soybean oil, mineral oil, sesame oil and the like, polyethylene glycols, glycerine, propylene glycol or other synthetic solvents. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfide; and agents for the adjustment of tonicity such as sodium chloride or dextrose are also envisioned.

The compositions can take the form of solutions, suspensions, emulsions, tablets, pills, capsules, powders, gels, creams, ointments, foams, pastes, sustained-release formulations and the like. The compositions can be formulated as a suppository, with traditional binders and carriers such as triglycerides, microcrystalline cellulose, gum tragacanth or gelatine. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences” by E. W. Martin, the contents of which are hereby incorporated by reference herein. Such compositions will contain a therapeutically effective amount of the peptide of the invention, preferably in a substantially purified form, together with a suitable amount of carrier so as to provide the form for proper administration to the subject.

An embodiment of the invention relates to a polypeptide presented in unit dosage form and are prepared by any of the methods well known in the art of pharmacy. In an embodiment of the invention, the unit dosage form is in the form of a tablet, capsule, lozenge, or wafer. In addition, in vitro assays may optionally be employed to help identify optimal dosage ranges. The precise dose to be employed in the formulation will also depend on the route of administration. Effective doses can be extrapolated from dose-response curves derived from in-vitro or in-vivo animal model test bioassays or systems.

In an embodiment of the invention, polypeptides are administered via oral route of administration.

For oral applications, the pharmaceutical composition may be in the form of drops, tablets, or capsules, which can contain any of the following ingredients, or compounds of a similar nature: a binder such as microcrystalline cellulose, gum tragacanth or gelatine; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

The compositions of the present invention are generally administered in the form of a pharmaceutical composition comprising at least one of the active components of this invention, e.g., the chimeric polypeptide, a cell comprising the chimeric polypeptide of the invention, together with a pharmaceutically acceptable carrier or diluent. Thus, the compositions of this invention can be administered either individually or together in any conventional oral, parenteral, or transdermal dosage form.

### Chimeric Polypeptides

In some embodiments, the first polypeptide comprises a heat labile toxin subunit B (LTB) polypeptide comprising the sequence:

(SEQ ID NO: 1)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKN,
or
an analog thereof having at least 80% sequence identity to the LTB,
as long as the analog has the activity of the LTB, e.g., insertion of the
second polypeptide across a cell membrane.

In some embodiments, the first polypeptide comprises a Cholera toxin B subunit (CTB), or an analog thereof having at least 80% sequence identity to the CTB, as long as the analog has the activity of the CTB, e.g., insertion of the second polypeptide across a cell membrane.

In some embodiments, the LTB functional analog comprises CTB.

In some embodiments, the first polypeptide and the second polypeptide are operably linked. In some embodiments, the first polypeptide and the second polypeptide are continuously linked, or contiguous. In some embodiments, the first polypeptide and the second polypeptide are linked via a linker. In some embodiments, the linker is a polypeptide linker. In some embodiments, a polypeptide linker is a dipeptide or longer.

In some embodiments, the second polypeptide comprises at least one a viral peptide. In some embodiments, a viral peptide is any peptide produced, secreted, or derived from a virus. In some embodiments, a viral peptide is a synthetic (e.g., recombinant) peptide substantially identical to a peptide naturally occurring within a virus.

In some embodiments, the viral peptide is a viral fusion peptide, wherein the fusion peptide comprises a plurality of viral derived peptides. In some embodiments, a plurality comprises at least 2, at least 3, at least 5, at least 7, at least 8, or at least 10, or any value and range there between. Each possibility represents a separate embodiment of the invention.

In one embodiment, the viral fusion peptide comprises two viral polypeptides. In some embodiments, a viral fusion peptide comprises a spike protein fused to a nucleocapsid protein.

As used herein, the term “fused” refers to a case wherein two distinct polypeptides are a single continuous chain of amino acids, e.g., a polypeptide comprising the amino acid sequence of both, one after the other.

In some embodiments, the fusion polypeptide is encoded by a single chimeric polynucleotide comprising the coding region of each viral gene operably linked to one another. In some embodiments, the fusion polypeptide is produced by expressing the aforementioned chimeric polynucleotide in a compatible expression system as discussed hereinbelow. In some embodiments, the fusion polypeptide is produced by ligating each of the distinct viral peptides so as to obtain a single fused polypeptide. In some embodiments, a linker is located between the distinct viral peptides.

In some embodiments, the viral peptide comprises a partial sequence or portion of a viral peptide, as long as the partial sequence or portion of the peptide comprises or consists of a defined or an intact structural motif or domain.

In some embodiments, the partial sequence or portion of the peptide is able to keep the polypeptide in a stable and/or soluble conformation. In some embodiments, the present invention is further directed to the selection of a viral peptide or a partial sequence thereof, as long as viral peptide or the partial sequence thereof maintain a stable and/or soluble conformation in vitro. In some embodiments, the present invention is further directed to the selection of a viral peptide or a partial sequence thereof, as long as viral peptide or the partial sequence thereof maintains a stable and/or soluble conformation in vivo. In some embodiments, the present invention is further directed to the selection of a viral peptide or a partial sequence thereof, as long as viral peptide or the partial sequence thereof maintain a stable soluble conformation induce immunogenic response of a host. In some embodiments, the present invention is further directed to the selection of a viral peptide or a partial sequence thereof, as long as viral peptide or the partial sequence thereof maintain a stable soluble conformation a subject administered with the viral peptide or the partial sequence thereof. In some embodiments, wherein a partial viral peptide sequence is selected as the second polypeptide of the chimeric polypeptide of the invention, the partial sequence is selected based on its structural similarity or homology to the structure of the full viral peptide. In some embodiments, the partial viral sequence peptide is a complete domain of the full viral peptide. In some embodiments, the partial viral sequence peptide has a structure or natively folds substantially similar the corresponding amino acids within the full viral peptide. The suitability of a partial viral sequence peptide to be used according to the herein disclosed methods, can be determined based on anyone of stability (e.g., biological half-life), predicted or determined structural similarity (e.g., bioinformatics based on resolved structures, x-ray diffraction, etc.), and solubility.

In some embodiments, a viral peptide is a viral spike protein, a viral nucleocapsid protein, a fusion peptide of both, or any fragment or domain thereof. In some embodiments, a viral peptide comprises the full-length amino acid sequence or a partial amino acid sequence of the viral peptide. In some embodiments, a viral peptide in an analog of a viral peptide having at least 80% sequence identity as long as the analog has the activity of the viral peptide, e.g., immunogenic antigen inducing IgG production.

As used herein, the term fragment refers to any amino acid sequence comprising 10 to 100, 10 to 200, 10 to 300, 10 to 400, 10 to 500, 10 to 600, or 10 to 650 amino acids derived from a viral spike protein or a viral nucleocapsid protein.

In some embodiments, the viral peptide is a domain derived from a viral spike protein or a viral nucleocapsid protein.

In some embodiments, the viral peptide is or comprises the receptor binding domain (RBD) of a viral spike protein.

In some embodiments, the spike protein comprises the amino acid sequence:

(SEQ ID NO: 2)
VNLTTRTQLPPAYTNSFTRGVYYPDKVFRSSVLHSTQDLFLPFFSNVTWFHAIHVSGING
TKRFDNPVLPFNDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNFKNLREFVF
KNIDGYFKIYSKHTPINLVRDLPQGFSALEPLVDLPIGINITRFQTLLALHRSYLTPGDSSS
GWTAGAAAYYVGYLQPRTFLLKYNENGTITDAVDCALDPLSETKCTLKSFTVEKGIYQ
TSNFRVQPTESIVRFPNITNLCPFGEVENATRFASVYAWNRKRISNCVADYSVLYNSASF
STFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQ
SYGFQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGIC
ASYQTQTNSPRRAR.

In some embodiments, the spike protein RBD comprises the amino acid sequence:

(SEQ ID NO: 8)
EFITNLCPFGEVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNGVGYQPY
RVVVLSFELLHAPATVCGPKKSTNLVKNKXVNFNFNGLTGT, wherein X
is cysteine or alanine.

In some embodiments, the spike protein RBD comprises the amino acid sequence:

(SEQ ID NO: 15)
ITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNGVGYQPY
RVVVLSFELLHAPATVCGPKKSTNL.

In some embodiments, the spike protein RBD comprises or consists of the amino acid sequence set forth in SEQ ID NO: 8 or any functional analog thereof having at least 50%, at least 60%, at least 70%, at least 80%, at least 90%, at least 95%, at least 97%, at least 99%, or 100% sequence identity thereto, or any value and range therebetween. Each possibility represents a separate embodiment of the invention.

In some embodiments, the spike protein or RBD thereof comprises any analog thereof having at least 50%, at least 60%, at least 70%, at least 80%, at least 90%, at least 95%, at least 97%, at least 99%, or 100% sequence identity to SEQ ID NO: 2 or SEQ ID Nos: 8, 12, 13, and 14, respectively.

In some embodiments, the spike protein comprises any analog having at least 70% sequence identity to SARS-COV Spike protein (e.g., UniProt Number: P59594).

In some embodiments, the spike protein comprises any analog having at least 70% sequence identity to MERS-COV Spike protein (e.g., UniProt Number: K9N5Q8).

In some embodiments, the spike protein RBD comprises the amino acid sequence:

(SEQ ID NO: 12)
ITNLCPFGEVFNATRFASVYAWNRKRISNCVADYSVLYNSASFSTFKCY
GVSPTKLNDLCFTNVYADSFVIRGDEVRQIAPGQTGNIADYNYKLPDDF
TGCVIAWNSNNLDSKVGGNYNYLYRLFRKSNLKPFERDISTEIYQAGST
PCNGVKGFNCYFPLQSYGFQPTYGVGYQPYRVVVLSFELLHAPATVCGP
KKSTNL.

In some embodiments, the spike protein RBD comprises the amino acid sequence:

(SEQ ID NO: 13)
ITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDF
TGCVIAWNSNNLDSKVGGNYNYRYRLFRKSNLKPFERDISTEIYQAGSK
PCNGVEGENCYFPLQSYGFQPTNGVGYQPYRVVVLSFELLHAPATVCGP
KKSTNL.

In some embodiments, the spike protein RBD comprises the amino acid sequence:

(SEQ ID NO: 14)
ITNLCPFDEVFNATRFASVYAWNRKRISNCVADYSVLYNLAPFFTFKCY
GVSPTKLNDLCFTNVYADSFVIRGDEVRQIAPGQTGNIADYNYKLPDDF
TGCVIAWNSNKLDSKVSGNYNYLYRLFRKSNLKPFERDISTEIYQAGNK
PCNGVAGFNCYFPLKSYSFRPTYGVGHQPYRVVVLSFELLHAPATVCGP
KKSTNL.

In some embodiments, the nucleocapsid protein comprises the amino acid sequence:

(SEQ ID NO: 3)
NNTASWFTALTQHGKEDLKFPRGQGVPINTNSSPDDQIGYYRRATRRIR
GGDGKMKDLSPRWYFYYLGTGPEAGLPYGANKDGIIWVATEGALNTPKD
HIGTRNPANNAAIVLQLPQGTTLPKGFYAEGS.

In some embodiments, the nucleocapsid protein comprises the amino acid sequence:

(SEQ ID NO: 4)
AAEASKKPRQKRTATKAYNVTQAFGRRGPEQTQGNFGDQELIRQGTDYK
HWPQIAQFAPSASAFFGMSRIGMEVTPSGTWLTYTGAIKLDDKDPNFKD
QVILLNKHIDAYKT.

In some embodiments, the nucleocapsid protein comprises the amino acid sequence:

(SEQ ID NO: 10)
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA.

In some embodiments, the nucleocapsid protein comprises any analog having at least 80% sequence identity to SEQ ID NO: 3, SEQ ID NO: 4, or SEQ ID NO: 10.

In some embodiments, the linker comprises an amino acid sequence of 2 to 10 amino acids. In some embodiments, the linker comprises 3 to 7 amino acids. In some embodiments, the linker comprises or consists of Serine and Glycine amino acid residues.

In some embodiments, the chimeric polypeptide comprises the sequence of:

(SEQ ID NO: 5)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKNGGSGGQCVNLTTRTQLPPAYTN
SFTRGVYYPDKVFRSSVLHSTQDLFLPFFSNVTWFHAIHVSGTNGTKRF
DNPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPL
VDLPIGINITRFQTLLALHRSYLTPGDSSSGWTAGAAAYYVGYLQPRTF
LLKYNENGTITDAVDCALDPLSETKCTLKSFTVEKGIYQTSNFRVQPTE
SIVRFPNITNLCPFGEVENATRFASVYAWNRKRISNCVADYSVLYNSAS
FSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHA
PATVCGPKKSTNLVKNKCVNFNFNGLTGTGVLTESNKKFLPFQQFGRDI
ADTTDAVRDPQTLEILDITPCSFGGVSVITPGTNTSNQVAVLYQDVNCT
EVPVAIHADQLTPTWRVYSTGSNVFQTRAGCLIGAEHVNNSYECDIPIG
AGICASYQTQTNSPRRAR.

In some embodiments, the chimeric polypeptide comprises the sequence of:

(SEQ ID NO: 6)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKNGGSGGNNTASWFTALTQHGKED
LKFPRGQGVPINTNSSPDDQIGYYRRATRRIRGGDGKMKDLSPRWYFYY
LGTGPEAGLPYGANKDGIIWVATEGALNTPKDHIGTRNPANNAAIVLQL
PQGTTLPKGFYAEGS.

In some embodiments, the chimeric polypeptide comprises the sequence of:

(SEQ ID NO: 7)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKNGGSGGAAEASKKPRQKRTATKA
YNVTQAFGRRGPEQTQGNFGDQELIRQGTDYKHWPQIAQFAPSASAFFG
MSRIGMEVTPSGTWLTYTGAIKLDDKDPNFKDQVILLNKHIDAYKT.

In some embodiments, the chimeric polypeptide comprises the sequence of:

(SEQ ID NO: 9)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKNGGSGGEFITNLCPFGEVENATR
FASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSK
VGGNYNYLYRLFRKSNLKPFERDISTEIYQAGSTPCNGVEGENCYFPLQ
SYGFQPTNGVGYQPYRVVVLSFELLHAPATVCGPKKSTNLVKNKXVNFN
FNGLTGT, wherein X is cysteine or alanine.

In some embodiments, the chimeric polypeptide comprises the sequence of:

(SEQ ID NO: 11)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKNGGSGGSDNGPQNQRNAPRITFG
GPSDSTGSNQNGERSGARSKQRRPQGLPNNTASWFTALTQHGKEDLKFP
RGQGVPINTNSSPDDQIGYYRRATRRIRGGDGKMKDLSPRWYFYYLGTG
PEAGLPYGANKDGIIWVATEGALNTPKDHIGTRNPANNAAIVLQLPQGT
TLPKGFYAEGSRGGSQASSRSSSRSRNSSRNSTPGSSRGTSPARMAGNG
GDAALALLLLDRLNQLESKMSGKGQQQQGQTVTKKSAAEASKKPRQKRT
ATKAYNVTQAFGRRGPEQTQGNFGDQELIRQGTDYKHWPQIAQFAPSAS
AFFGMSRIGMEVTPSGTWLTYTGAIKLDDKDPNFKDQVILLNKHIDAYK
TFPPTEPKKDKKKKADETQALPQRQKKQQTVILLPAADLDDFSKQLQQS
MSSADSTQA.

In some embodiments, the chimeric polypeptide comprises the sequence of:

(SEQ ID NO: 16)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKNEFITNLCPFGEVFNATRFASVY
AWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNY
NYLYRLFRKSNLKPFERDISTEIYQAGSTPCNGVEGENCYFPLQSYGFQ
PTNGVGYQPYRVVVLSFELLHAPATVCGPKKSTNL.

Id no: In some embodiments, the chimeric polypeptide comprises the sequence of:

(SEQ ID NO: 17)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKNITNLCPFGEVENATRFASVYAW
NRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNY
LYRLFRKSNLKPFERDISTEIYQAGSTPCNGVEGENCYFPLQSYGFQPT
NGVGYQPYRVVVLSFELLHAPATVCGPKKSTNL.

In some embodiments, the second polypeptide comprises an immunogenic antigen of a pathogenic virus, bacterium, or a fungus.

As used herein, the term “immunogenic” refers to any compound inducing or priming the immune system of a host subject to produce immunoglobulins targeting the immunogenic antigen.

### Definitions

The term “amino acid” as used herein means an organic compound containing both a basic amino group and an acidic carboxyl group.

The term “amino acid residue” as used herein refers to the portion of an amino acid that is present in a peptide.

The term “peptide bond” means a covalent amide linkage formed by loss of a molecule of water between the carboxyl group of one ammo acid and the ammo group of a second ammo acid.

The terms “peptide”, “polypeptide” and “protein” as used herein encompass native peptides, peptidomimetics (typically including non-peptide bonds or other synthetic modifications) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

The term “peptide”, “polypeptide” and “protein” are used herein interchangeably.

One of skill in the art will recognize that individual substitutions, deletions or additions to a peptide, or protein sequence which alters, adds, or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to an aspartic acid (D).

As used herein, the phrase “conservative substitution” also includes the use of a chemically derivatized residue in place of a non-derivatized residue provided that such peptide displays the requisite function as specified herein.

Peptide derivatives can also include side chain bond modifications, including but not limited to —CH2—NH—, —CH2—S—, —CH2—S=0, OC—NH—, —CH2—O—, —CH2—CH2—, S=C—NH—, and —CH═CH—, and backbone modifications such as modified peptide bonds. Peptide bonds (—CO—NH—) within the peptide can be substituted, for example, by N-methylated bonds (—N(CH3)—CO—); ester bonds (—C(R)H—C—O—O—C(R)H—N); ketomethylene bonds (—CO—CH2—); a-aza bonds (—NH—N(R)—CO—), wherein R is any alkyl group, e.g., methyl; carba bonds (—CH2—NH—); hydroxyethylene bonds (—CH(OH)—CH2—); thioamide bonds (—CS—NH); olefmic double bonds (—CH═CH—); and peptide derivatives (—N(R)—CH2—CO—), wherein R is the “normal” side chain, naturally presented on the carbon atom. These modifications can occur at one or more of the bonds along the peptide chain and even at several (e.g., 2-3) at the same time.

As used herein, the term “chimera” encompasses any conjugate comprising two or more moieties, wherein the two or more moieties are bound to one another either directly or indirectly, and wherein the moieties are either derived from distinct origins or are not naturally bound to one another. In some embodiments, the two or more moieties have: distinct functions, originate or derived from different genes, peptides, genomic regions, or species, distinct chemical classification (e.g., a peptide and a polynucleotide, as exemplified herein).

In some embodiments, the chimera of the invention comprises the peptide of the invention bound directly or indirectly to an agent, wherein the agent is selected from: a nucleotide, an oligonucleotide, a polynucleotide, an amino acid, a peptide, a peptide, a protein, a small molecule, a synthetic molecule, an organic molecule, an inorganic molecule, a polymer, a synthetic polymer, or any combination thereof.

As used herein, the term “directly” refers to cases wherein the peptide of the invention is bound to the agent in a covalent bond.

As used herein, the term “indirectly” refers to cases wherein each of the peptide of the invention and the agent are bound to a linker or a spacing element and not directly to one another. In some embodiments, the peptide is covalently bound to the linker. In some embodiments, the agent is either covalently or non-covalently bound to the linker.

As used herein, the term “covalent bond” refers to any bond which comprises or involves electron sharing. Non-limiting examples of a covalent bond include, but are not limited to: peptide bond, glyosidic bond, ester bond, phosphor diester bond.

As used herein, the term “non-covalent bond” encompasses any bond or interaction between two or more moieties which do not comprise or do not involve electron sharing. Non-limiting examples of a non-covalent bond or interaction include, but are not limited to, electrostatic, x-effect, van der Waals force, hydrogen bonding, and hydrophobic effect.

The term “linker” refers to a molecule or macromolecule serving to connect different moieties of the chimera, that is the peptide of the invention and the agent. In one embodiment, a linker may also facilitate other functions, including, but not limited to, preserving biological activity, maintaining sub-units and domains interactions, and others.

In another embodiment, a linker may be a monomeric entity such as a single amino acid. In another embodiment, amino acids with small side chains are especially preferred, or a peptide chain, or polymeric entities of several amino acids. In another embodiment, a peptide linker is 2 to 30 amino acids long, 2 to 25 amino acids long, 4 to 23 amino acids long, 4 to 20 amino acids long, 5 to 22 amino acids long, or 2 to 28 amino acids long. Each possibility represents a separate embodiment of the invention. In another embodiment, a peptide linker is at least 6 amino acids long, at least 8 amino acids long, at least 10 amino acids long, at least 12 amino acids long, at least 15 amino acids long, at least 17 amino acids long, at least 20 amino acids long, at least 22 amino acids long, at least 25 amino acids long, at least 27 amino acids long, or at least 30 amino acids long, or any value and range therebetween. Each possibility represents a separate embodiment of the invention. In one embodiment, a linker may be a nucleic acid encoding a small peptide chain. In another embodiment, a linker encodes a peptide linker of 6 to 30 amino acids long, 6 to 25 amino acids long, 7 to 23 amino acids long, 8 to 20 amino acids long, 10 to 22 amino acids long, or 12 to 28 amino acids long. Each possibility represents a separate embodiment of the invention. In another embodiment, a linker encodes a peptide linker of at least 6 amino acids long, at least 8 amino acids long, at least 10 amino acids long, at least 12 amino acids long, at least 15 amino acids long, at least 17 amino acids long, at least 20 amino acids long, at least 22 amino acids long, at least 25 amino acids long, at least 27 amino acids long, or at least 30 amino acids long, or any value and range therebetween. Each possibility represents a separate embodiment of the invention.

In some embodiments, the peptide of the invention may be attached or linked to an agent via a chemical linker. Chemical linkers are well known in the art and include, but are not limited to, dicyclohexylcarbodiimide (DCC), N-hydroxysuccinimide (NHS), maleiimidobenzoyl-N-hydroxysuccinimide ester (MBS), N-ethyloxycarbonyl-2-ethyloxy-1,2-dihydroquinoline (EEDQ), N-isobutyloxy-carbonyl-2-isobutyloxy-1,2-dihydroquinoline (IIDQ).

In another embodiment, the linker may be biodegradable such that the peptide of the invention is further processed by hydrolysis and/or enzymatic cleavage inside cells. In some embodiments, a readily cleavable group include acetyl, trimethylacetyl, butanoyl, methyl succinoyl, t-butyl succinoyl, methoxycarbonyl, ethoxycarbonyl, benzoyl, 3-aminocyclohexylidenyl, and the like.

In some embodiments, a peptide linker has an electric charge at a pH ranging from 6.5 to 8.

In some embodiments, the linker has a positive electric charge. In some embodiments, the linker has a negative electric charge.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, and “having” are interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

As used herein, the term “about” when combined with a value refers to plus and minus 10% of the reference value. For example, a length of about 1,000 nanometres (nm) refers to a length of 1000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

TABLE 1
Experimental time schedule

| | | Group | Group | Group | Group | Group | Group | | | Group |
|---|---|---|---|---|---|---|---|---|---|---|
| Day | Activity | 1 | 2 | 3 | 4 | 5 | 6 | Day | Activity | 7 |
| −2 | Baseline | + | + | + | + | + | + | | | |
| | bleeding for | | | | | | | | | |
| | serum | | | | | | | | | |
| 0 | 1st Priming- | + | + | + | + | + | + | | | |
| | Commercial | | | | | | | | | |
| | S + Adjuv SC | | | | | | | | | |
| 14 | Feces for IgA | + | + | | | | | | | |
| 15 | 1^{st} Boost- | + | | | | | | | | |
| | MigVax-101 | | | | | | | | | |
| | PO | | | | | | | | | |
| | 1^{st} Boost- | | + | | | | | | | |
| | Commercial S | | | | | | | | | |
| | SC | | | | | | | | | |
| | 2^{nd} Priming- | | | + | + | + | + | | | |
| | Commercial S | | | | | | | | | |
| | SC | | | | | | | | | |
| | | | | | | | | −2 | Baseline | + |
| | | | | | | | | | bleeding for | |
| | | | | | | | | | serum | |
| 28 | Feces for IgA | | | + | + | + | + | | | |
| 29 | 1^{s} Boost- | | | + | | | + | 0 | 1^{st} | + |
| | MigVax-101 | | | | | | | | Priming- | |
| | PO | | | | | | | | Commercial | |
| | | | | | | | | | S + Adjuv | |
| | | | | | | | | | SC | |
| | 1^{st} Boost- | | | | + | | | | | |
| | Commercial S | | | | | | | | | |
| | SC | | | | | | | | | |
| | Neg control | | | | | + | | | | |
| | boost-DP | | | | | | | | | |
| | Buffer PO | | | | | | | | | |
| | Feces for IgA | + | + | | | | | | | |
| | BAL | + | + | | | | | | | |
| | Serology, | + | + | | | | | | | |
| | pNT, | | | | | | | | | |
| | Cytokines | | | | | | | | | |
| | (spleen cell | | | | | | | | | |
| | ICS) | | | | | | | | | |
| | Termination | + | + | | | | | | | |
| | Feces for IgA | | | | | | | 11 | | + |
| 42 | 2^{nd} Boost- | | | | | | + | 14 | | |
| | MigVax-101 | | | | | | | | | |
| | PO | | | | | | | | | |
| | Neg control | | | | | + | | | Neg control | + |
| | boost-DP | | | | | | | | boost-DP | |
| | buffer | | | | | | | | buffer | |
| | Feces for IgA | | | + | + | + | | | | |
| | BAL | | | + | + | | | | | |
| | Serology, | | | + | + | + | | | | |
| | pNT, spleen | | | | | (serum | | | | |
| | ICS for | | | | | collection | | | | |
| | Cytokines | | | | | only) | | | | |
| | Termination | | | + | + | | | | | |
| 56 | Feces for IgA | | | | | + | + | 28 | Feces for | + |
| | | | | | | | | | IgA | |
| | BAL | | | | | + | + | | BAL | + |
| | Serology, | | | | | + | + | | Serology, | + |
| | pNT, spleen | | | | | | | | pNT, spleen | |
| | ICS for | | | | | | | | | |
| | Cytokines | | | | | | | | ICS for | |
| | | | | | | | | | Cytokines | |
| | Termination | | | | | + | + | | Termination | + |

### Ethical Committee

This study is performed following an application-form review by the “Committee for Ethical Conduct in the Care and Use of Laboratory Animals”, after receiving their approval that the Study complies with the rules and regulations set forth. The Ethical committee approval number for this Study is: IL-20-10-450.

Animal Welfare According to: Animal Welfare Law (Animal Studies)—1994 (State of Israel); Guide for the Care and Use of Laboratory Animals, the Institute of Laboratory Animal Research (ILAR); Guidelines of the National Institute of Health (NIH); and Association for Assessment and Accreditation of Laboratory Animal Care (AAALAC)

TABLE 2
Test system characterization

| | | | Number of | Bacteriological | Approximate |
|---|---|---|---|---|---|
| Species | Strain | Sex | Animals | Status | Age (weeks) |
| Rat | SD | Male | 25 | Specific pathogen- | 8 |
| | | | | free (SPF) | |
| Rat | SD | Female* | 25 | Specific pathogen- | 8 |
| | | | | free (SPF) | |

*Females are nulliparous and non-pregnant

### Housing

Animal handling is performed according to guidelines of the NIH and the AAALAC. Animals are housed in IVCs in dedicated HVAC in the SPF animal facility, equipped with a pressurized climatic system (ISO 8 certified). Animals are kept in solid bottom polysulfone cages, filled with bedding material.

### Environment Conditions

Mean temperature and humidity (RH) are maintained at 22±3° C. and 50±20%, respectively. Temperature and RH are monitored continuously and recorded in 5 minutes intervals. The facility has no exposure to outside light and is maintained on automatic alternating light cycles of 12L:12D.

### Water and Food

Water and commercial rodent food are supplied ad libitum during the acclimatization and study period. Bottles with stainless steel sipper-tubes and tap water are autoclaved before use.

### Experimental Design

### Duration of the Experimental Period

Fifty-eight (58) days, starting on Day −b 2.

### Animal Receipt and Acclimatization

Upon arrival, the animals are tagged, weighed, and assigned into groups. The animals have at least 5 days of acclimatization to the facility conditions, prior to study initiation. During this period, the health status of the animals is monitored.

### Group Assignment

Animals are assigned into treatment groups based on their body weight, thereby creating homogenous groups.

### Test Items Administration

Time points: Days 0, 15, 29 and 42 according to Table 1.

Route of administration: Oral feeding (PO) or subcutaneous (SC), according to Tables 1 and 3.

Equipment: The administration is through a disposable 1 ml syringe and 27G needle (SC) or reusable 20G gavage needle (PO).

Administration groups: The groups of administration are specified in Table 3.

Procedure: Rats are SC injected with S1 protein and an adjuvant as 1- or 2-dose priming regimen, 2-weeks apart. Then a booster regimen is administered, either SC injected S1, or oral MigVax-101 or control DP buffer, in 1- or 2-dose regimens, 2-weeks apart.

For the oral administration, dose is dripped gently on the top of the tongue, right beyond the lip line. This is done with a syringe attached to gavage cannula (20G) over 30 seconds (verified using timer).

Administration schedule is presented in Table 1.

TABLE 3
Groups of administration

| | | | | | Termination | Injection | Oral |
|---|---|---|---|---|---|---|---|
| | | | | | Day (in overall | Volume | volume |
| Group | (n) | Sex | Priming | Boost | experiment) | (μl) | (μl) |
| 1 | 4 | M | SC Comm S + | PO MigVax-101× 1 | 29 | 200 | 200 |
| | 5 | F | Adjuv × 1 | | | | |
| 2 | 5 | M | SC Comm S + | SC Comm S × 1 | 29 | 200 | 200 |
| | 4 | F | Adjuv × 1 | | | | |
| 3 | 4 | M | SC Comm S + | PO MigVax-101 × 1 | 42 | 200 | 200 |
| | 5 | F | Adjuv × 2 | | | | |
| 4 | 5 | M | SC Comm S + | SC Comm S × 1 | 42 | 200 | 200 |
| | 4 | F | Adjuv × 2 | | | | |
| 5 | 3 | M | SC Comm S + | PO DP Buffer × 2 | 56 | 200 | 200 |
| | 2 | F | Adjuv × 2 | | | | |
| 6 | 4 | M | SC Comm S + | PO MigVax-101 × 2 | 56 | 200 | 200 |
| | 5 | F | Adjuv × 2 | | | | |
| 7 | 4 | M | SC Comm S + | PO DP Buffer × 1 | 28 (56) | 200 | 200 |
| | 5 | F | Adjuv × 1 | | | | |

### Clinical Signs

Animals are observed twice a day for morbidity and mortality, and daily for general clinical signs such as changes in the skin, fur, eyes, nose, mouth, head, respiration, urine, feces, locomotor, and overall wellness.

### Body Weight

Animals are weighed, at the following time points: On arrival; At least once a week during the study period; and on termination.

### Humane Endpoints

An animal showing one of the following humane endpoints, is humanely euthanized and subjected to the termination procedure: Loss of 20% of body weight from baseline weight; Major organ failure or medical conditions unresponsive to treatment; Clinical or behavioural signs of acute unrelievable stress or significant chronic stress or distress that are unresponsive to appropriate intervention; and one of the following abnormalities persisting for 24 hours: Inactivity or hyper-activity; Labored breathing; Hunched posture; Piloerection/matted fur; Signs of dehydration; Abnormal vocalization when handled; Anorexia; and one or more unresolvable skin ulcers.

### Mortality

If a study animal dies, the time of death is recorded as precisely as possible. The animal is subjected to the termination procedure, if possible.

### Serum Collection Only

Time points: Days −2 (all animals) and 42 (Group 5) according to Table 1.

Serum (needs clot time):

Venous blood is collected from the retroorbital sinus;

Collect at least 800 μl of blood into yellow top gel tubes;

Allow samples to clot for 30 minutes at room temperature;

Centrifuge at 22° C. and 3,000g for 10 minutes;

Draw the serum into a tube and distribute equal volumes (at least 150 μl) into 3 Eppendorf vials; and

Transfer immediately to −70° C. freezer.

### Feces Collection

Time points: Days 14, 28/29, 42 and 56, according to Table 1.

Procedure: Feces are collected from each animal according to the schedule in Table 1. Animals are transferred to metabolic cages and smooth pieces of feces are inserted into an Eppendorf tube, and transferred immediately to −20° C. The samples are of at least 2 pieces of feces from each animal. At termination, feces are collected directly from the rectum or colon.

### Termination

Time Point: Days 29, 42 and 56 according to Table 1.

Terminal Anesthesia—Animals are anesthetized by intraperitoneal (IP) injection of ketamine-xylazine cocktail.

Terminal Specimen Collection and Handling.

Whole blood is collected from each animal, as follows:

Serum (needs clot time);

Collect at least 500 μl of blood into yellow top gel tubes;

Allow samples to clot for 30 minutes at room temperature;

Centrifuge at 22° C. and 3,000 g for 10 minutes;

Draw the serum into a tube and distribute equal volumes into 3 Eppendorf vials; and Transfer immediately to −70° C. freezer

### BAL

Extract BAL from lungs, by one slow wash with 1.5 ml. The recollected volume (˜1 ml) is transferred to an Eppendorf tube containing 0.5 ml PBS. With this volume another 2 washes are performed. Approximately 1.5 ml of PBS, per each animal, is obtained as final BAL volume per animal;

Centrifuge samples at 22° C., 400×g for 5 minutes;

Transfer the supernatant into new tubes; and

Freeze immediately at −70° C.

### Spleen

Excise the whole spleens into 15 ml tubes.

Add immediately sterile PBS×1 at 2-8° C. well covering the entire spleen.

### EXAMPLE 1

### Oral Boosting Using RBD After S1 Injection Induces Production of Neutralizing Antibodies

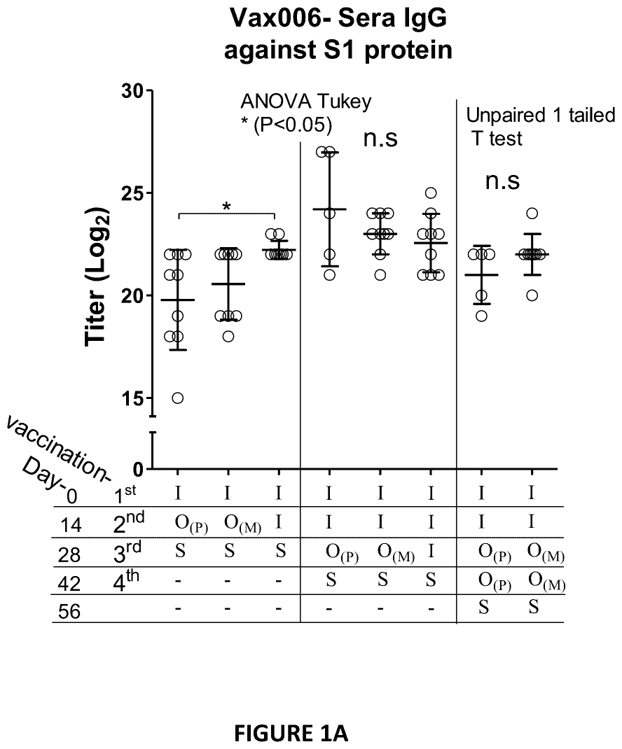

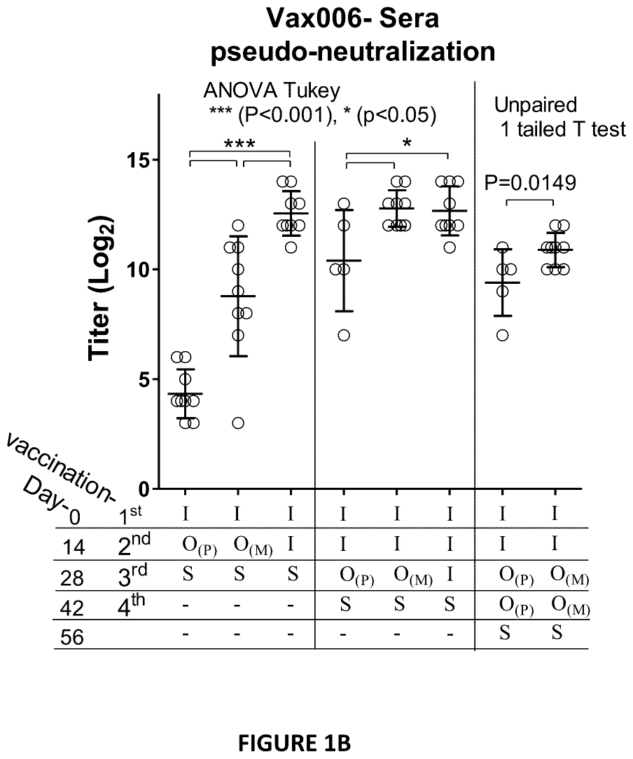

The inventors showed that after 28 days, oral boost with MigVax after one injection did not significantly increase anti-S1 IgG as compared to placebo (FIG. 1A). Nonetheless, the oral boosting did strongly and significantly increase the levels of neutralizing antibodies (FIG. 1B). Further, after 42 days, oral boost after two injections induced neutralizing antibody levels comparable to injection (FIG. 1B). After 56 days, two oral boosts significantly increased neutralizing antibody levels, compared to placebo (FIG. 1B).

### EXAMPLE 2

### Oral Boosting Increases Anti S1 IgA in BALF

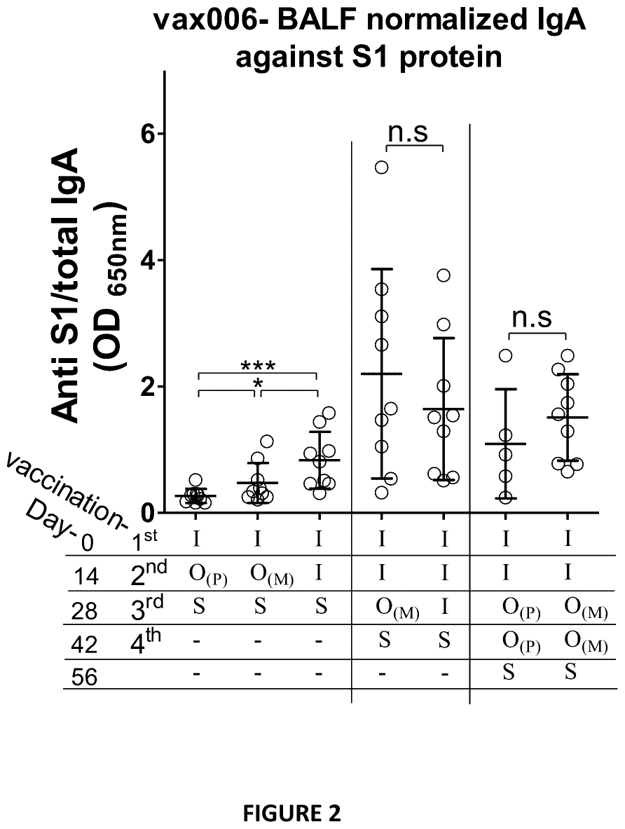

The inventors showed that after 28 days, after priming with one S1 injection, boost with one oral MigVax101 administration significantly increased anti-S1 IgA levels, compared to oral placebo administration (FIG. 2). Further, after 42 days, one oral MigVax101 boost post two injections resulted with IgA levels at least as high as obtained with three injections (FIG. 2). After 56 days, two oral MigVax101 administrations after two injections were shown to increase anti-S1 IgA, compared to two oral placebo administrations (FIG. 2).

Based on the above, the inventors suggest to orally administer the S1 RBD (as a “booster”) to a subject previously been administered by means of injections with the S1, so as to improve the immunization reaction in the subject, production of neutralizing antibodies. Such oral administration clearly provides an alternative and/or improved practice of vaccination with improved compliance, e.g., for young subjects, e.g., kids.

### EXAMPLE 3

### Vaccine Protein Composition in a Heterologous Setting

### Experiment Design

Rats are SC injected with S1 with complete/incomplete Freund's adjuvant, 2-dose priming regimen, 2-weeks apart (Table 4).

One booster dose is administered 6 weeks post 2 doses of priming injections: oral MigVax-101 in various compositions, SC SI as positive control (with incomplete Freund's adjuvant), or oral placebo as negative control.

Oral placebo dose consists of the vaccine buffer: 50 mM Phosphate buffer, pH=7.2, 150 mM NaCl, 0.1% Tween 20, 15% Glycerol.

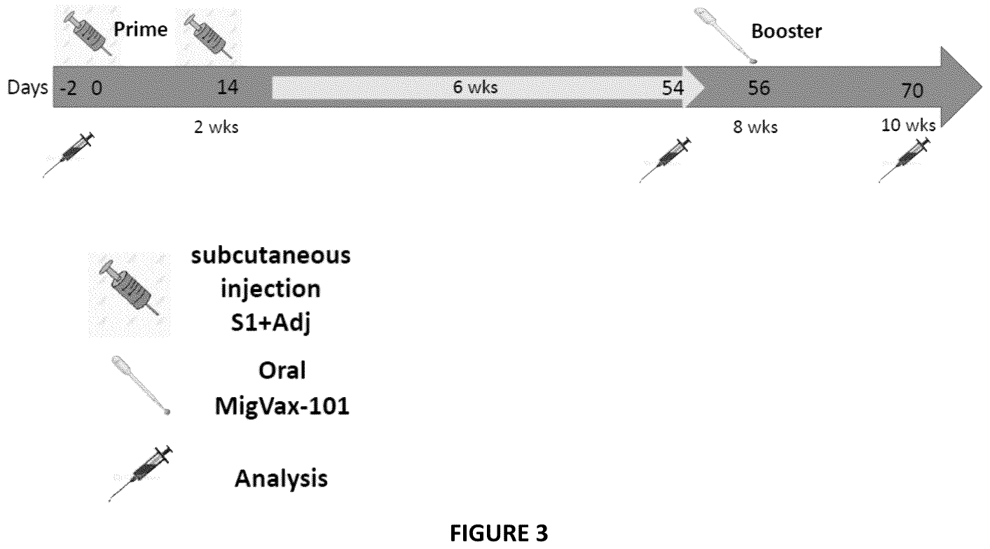

TABLE 4
Heterologous boost study outline (see also FIG. 3)

| | | | | |
|---|---|---|---|---|
| Days | 0, 14 | 15-55 | 56 [8] | 70 [10] |
| [weeks] | [0, 2] | [3-7] | | |
| Group #¹ | Priming | Lag | Boost | Length |
| | (×2) | period | | of study |
| | | 6 weeks | | (days) |
| 1 | S1 | | RBD + LTB-NN + LTB-NC + | 70 |
| | | | LTB | |
| 2 | | | RBD + LTB-NC | |
| 3 | | | RBD + LTB-NC X 2 | |
| 4 | | | RBD + LTB NC X 3 | |
| 5 | | | RBD + LTB-NC +LTB | |
| 6 | | | RBD + LTB-NC X 2 + LTB | |
| 7 | | | RBD + LTB-NC X 3 + LTB | |
| 8 | | | Placebo PO | |
| 9 | | | SC S1 | |
| 10 | | | RBD* + LTB-NC X 2 | |
| 11 | | | RBD* + LTB-NC X2 + LTB | |

¹each group consists of 10 animals, 5 males and 5 females
RBD—Receptor Binding Domain;
LTB—Heat-labile toxin B (E-coli);
NC—SARS-COV-2 Nucleocapsid protein, C-terminal;
PO—per Os;
S1—SARS-COV-2 Spike protein, S1 part;
RBD*—Diadyc manufactured

TABLE 5
Vaccine components

| | Amount (μg) | | |
|---|---|---|---|
| | ×1 | ×2 | ×3 |
| LTB-NC | 70 | 140 | 210 |

| | Amount (ug) |
|---|---|
| RBD | 90 |
| LTB | 35 |
| LTB-NN | 70 (35 μg LTB, |
| | 35 μg NN) |

### Laboratory Tests

Immunogenicity—Systemic Humoral: Blood is withdrawn in a volume of ˜1.0 ml for serum preparation.

Schedule of bleeding: Pre-vaccination (Day -2, only representative 15 rats in total are bled), 2 days pre-boost (Day 54), and on termination, Day 70. Serum is assayed by ELISA for IgG levels for anti-S1 (or RBD, TBD), and anti-LTB. An in vitro neutralization test for anti-S IgG is performed.

Immunogenicity—Mucosal: Wet feces and Lung wash fluid are collected on termination for IgA evaluations (total IgA, specific anti-S1 IgA; exploratory option—for BALF samples with highest anti-S1 levels—IgA neutralization assay).

For immunogenicity studies, serum are isolated and aliquoted into 3 vials of at least 120 μl each: 1 vial for SmartAssays (anti-S1 or RBD IgG), 1 vial for Central Virology Laboratories (neutralization) and 1 vial for MigVax (anti-LTB). Sera are transferred frozen on dry ice.

Basic Safety Evaluations: include surveillance for body weight and body temperature, at various time points according to Table 6.

TABLE 6
Safety and Immunological Evaluations
Safety Evaluations

| | |
|---|---|
| Mortality/ | Daily |
| Morbidity | |
| Clinical Signs | Daily |
| Body Weight: | Body weight is assessed during acclimation for all |
| | groups. |
| | Body weight is assessed for all groups once a |
| | week and 3 times in the 2 weeks following boost |
| | dose, and on termination. |

Immunological evaluation

| | |
|---|---|
| Serum for | Blood is collected from representative 15 rats pre- |
| antibody | prime S injection, Day −2, from all groups prior to |
| | boost (Day 54) and at sacrifice (Day 70). |
| Feces for | Feces are collected from all groups at sacrifice and |
| antibody | kept in case no IgA can be detected in BALF. |
| Removal and | Lungs are collected at sacrifice. Lungs are washed |
| washing of | with PBS through the trachea to obtain mucosal |
| Lungs | fluid extraction for mucosal IgA quantification. |
| | Lung fluid is frozen and kept at −20° C. |

Immunogenicity assessment

| | |
|---|---|
| Antibody | Geometric Mean Titer (GMT) for anti-RBD (or |
| Analysis | anti-S) |
| | Fold-change of IgG Antibody titer against RBD |
| | or anti-S in animals post boost with MigVax- |
| | 101 compared to each animal pre-boost (post |
| | two S1 injections), positive control (SC S1 |
| | administration) and compared to placebo. |
| Neutralization | Anti-S IgG antibodies ability to neutralize |
| assay | pseudovirus in vitro |
| | Geometric Mean Titer (GMT) for pseudo- |
| | neutralization |
| | Fold-change of pseudo-neutralization titer |
| | comparing pre- to post-boost titers and controls. |
| | Mean Fold-increase pseudo-neutralization titers |
| | as above. |
| Anti-LTB | Anti-LTB IgG levels are tested post boost, as a |
| | mean for controlling for MigVax-101 technical |
| | oral administration |
| Mucosal | Mucosal anti-S IgA response are determined by |
| immunity | ELISA on BALF. Feces are analyzed if no IgA is |
| | detected in BALF. BALF is collected on sacrifice, |
| | Day 70 from all groups. Highest anti-S1-IgA |
| | BALF may also be tested for IgA neutralization |
| | titers (exploratory) |
| Anti- | Anti-N IgG levels may be assessed, as exploratory |
| Nucleocapsid | measure, TBD |

### Statistical Considerations

Experiment aims are: (i) to determine the need for free LTB for optimal immunogenicity (neutralization); and (ii) to assess the total dose of free/fused LTB needed in the vaccine.

TABLE 7
Experimental groups

| | |
|---|---|
| 1 | RBD + LTB-NN + LTB-NC + LTB |
| 2 | RBD + LTB-NC |
| 3 | RBD + LTB-NC X 2 |
| 4 | RBD + LTB NC X 3 |
| 5 | RBD + LTB-NC + LTB |
| 6 | RBD + LTB-NC X 2 + LTB |
| 7 | RBD + LTB-NC X 3 + LTB |
| 8 | Placebo PO |
| 9 | S1 |

In order to achieve study aims, the inventors make the following analyses:

Initially, that group 1 is not different than group 6;

That group 8 is different from all other groups;

Whether group 9 is similar to any of groups 1-7;

In order to assess whether free LTB is needed for optimal immunological effect:

Is group 3 different from group 5;

Is group 4 different from group 6.

In order to assess what the optimal LTB dose should be: Assess blocks 2-4 and 5-7 separately (or which of them that is relevant, after assessing the need in free LTB)—the minimum dose of LTB-NC in each of the blocks, that shall cause optimal immunological response (neutralization).

### EXAMPLE 4

### Topical boosting using vaccine protein composition

Vaccine protein composition as disclosed herein is provided topically to previously vaccinated subjects.

Blood tests: 5 ml of blood are collected from each subject, on days 0, 7, 14, and 21 post oral administration. Sera are separated and preserved frozen till use.

Topical administration includes: RBD (45 μg), LTB-NC (70 μg), and LTB (17 μg). Reducing adverse effects is achieved by administering the topical vaccine in two consecutive days, each including half a dose.

Administration location and application: a surface area with a diameter of 5 cm on the forearm is wiped with a sandpaper about 10-15 times. Thereafter, the surface area is further cleaned with sterile water-soaked gauze, and dried. One hundred and 25 (125) μl including half a dose of the oral vaccine are dripped on the treated skin surface area. After application, the area is either sealed or left exposed till the entire topical vaccine dose is absorbed into the skin.

Efficacy:

Antibodies—enzyme linked immunosorbent assay (ELISA) is used to determine IgG, IgA specifically targeting LTB, N, and the RBD.

The following tests are performed (e.g., in the event that the levels of the above-mentioned antibodies are increased): neutralization (in vitro), and cellular tests (e.g., ELISPOT).

Following two topical applications of the vaccine protein composition as disclosed herein, the level of neutralizing antibodies is expected to increase by about 20-70%. Following a third topical vaccination, to be performed, for example after two weeks, neutralization (can be demonstrated for example in an in vitro test), is expected to reach 70-100%, thus comparable to an injection control (“positive control”).

### EXAMPLE 5

### Vaccine Protein Composition Reduces Infectivity of a Coronavirus in an Avian Model

Experimental groups are disclosed hereinbelow in Table 8.

TABLE 8
Experimental groups

| Group | N | Vaccine | Challenge |
|---|---|---|---|
| 1 | 3 | Caye broth | − |
| | | (negative control) | |
| 2 | 7 | Caye broth (positive | + |
| | | control) | |
| 3 | 9 | WT E. coli* | + |
| 4 | 7 | E. coli expressing | + |
| | | subunit proteins | |
| | | LS1& | |
| 5 | 13 | Mix of E. coli | + |
| | | expressing subunit | |
| | | proteins LNC and | |
| | | LNN† | |
| 6 | 13 | Mix of E. coli | + |
| | | expressing subunit | |
| | | proteins LS1, LNC | |
| | | and LNN | |
| 7 | 11 | Commercial | + |
| | | inactivated M41 | |

*WT: Wild type-Negative control E. coli that contains no plasmid
&LS1: Spike 1 protein fused to LTB
†LNC: N-terminal part of IBV N protein fused to LTB,
LNN: C-terminal part of IBV N protein fused to LTB

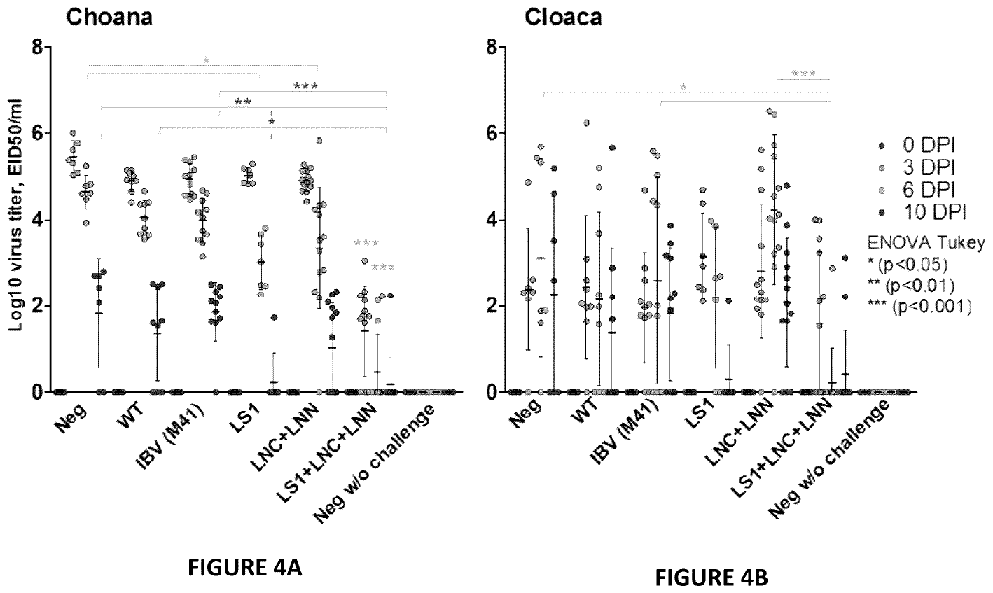

The concentrations of the virus shed over time from the choana and the trachea following challenge with live, virulent IBV are presented in FIG. 4. Virus concentrations in the group vaccinated with E. coli expressing LS1+LNC+LNN decreased significantly in the choana from day 0 to day 10 post-challenge and were significantly lower than in all other challenged groups. More specifically, on day 3 post-challenge, virus shedding from the choana was about 1.7 Log10 EID50/ml in the birds vaccinated with LS1+LNC+LNN, and about 5 Log10 EID50/ml in all other treatment groups. On days 6 and 10 post-challenge, virus titres for this group dropped below 1 Log10 EID50/mL. In all other challenged groups, titres of shed virus in the choana decreased only slightly by day 6. On day 10 post-challenge, only chicks vaccinated with LS1 or LS1+LNC+LNN showed shedding similar to that of the non-challenged negative control group. In all other groups, titres dropped below 2 Log10 EID50/ml, with high variability among the birds of each group. Due to this large variance within groups, statistical differences between groups were small. Nevertheless, titres of viruses shed on day 6 by chicks vaccinated with LS1+LNC+LNN were significantly lower than those found in chick groups vaccinated with, IBV (M41) or LNC+LNN control groups.

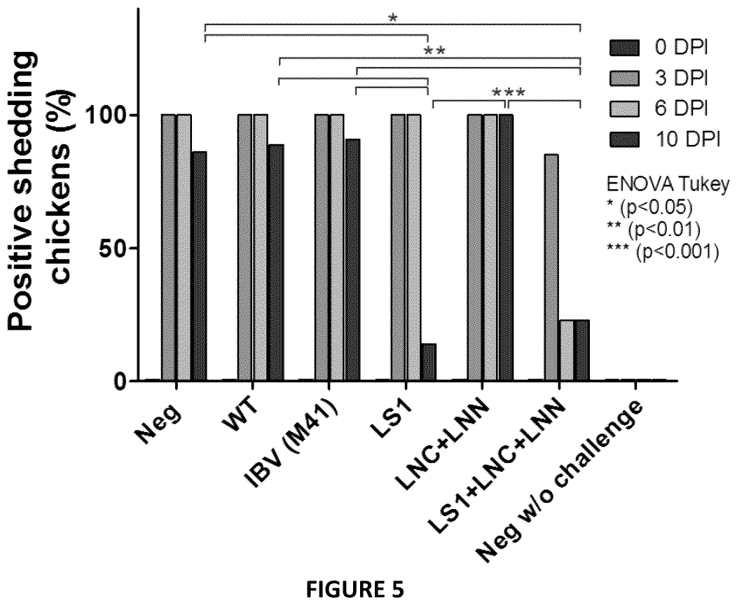

A time-course analysis of viral shedding from the choana or cloaca following challenge (day 0), showed that all vaccinated birds shed IBV 3 days after the challenge, apart from the group vaccinated with LS1+LNC+LNN, in which only 85% were shedders (FIG. 5). By 6 days post-challenge, only 23% of the LS1+LNC+LNN-vaccinated chickens were IBV shedders, while all bird in all other groups remained positive. On day 10 post-challenge, only 14% and 23% of the chickens vaccinated with LS1 or LS1+LNC+LNN, respectively, were shedders, a significantly lower percentage than the percentages found in the other test and control groups. In contrast, all of the LNC+LNN-vaccinated chicks continued to shed IBV until day 10 post-challenge.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for increasing immunization efficiency of a subject to a pathogen, the method comprising administering a therapeutically effective amount of a pharmaceutical composition comprising:
a. a heat labile toxin subunit B (LTB) polypeptide or a functional analog thereof; and
b. an immunogenic polypeptide derived from said pathogen,
thereby, increasing immunization efficiency of a subject to the pathogen.

2. The method of claim 1, wherein said administering comprises: orally administering, topically administering, or both.

3. The method of claim 1, wherein said LTB comprises the amino acid sequence:
(SEQ ID NO: 1)
MGNKVKCYVLFTALLSSLYAHGAPQTITELCSEYRNTQIYTINDKILSY
TESMAGKREMVIITFKSGETFQVEVPGSQHIDSQKKAIERMKDTLRITY
LTETKIDKLCVWNNKTPNSIAAISMKN.

4. The method of claim 1, wherein said subject is characterized by having above a predetermined threshold of immunoglobulins having specific binding affinity to said immunogenic polypeptide.

5. The method claim 1, further comprising a step preceding said administering comprising determining said subject comprises above a predetermined threshold of said immunoglobulins having specific binding affinity to said immunogenic polypeptide.

6. The method of claim 1, wherein said subject is a subject who previously diagnosed as being infected with said pathogen.

7. The method of claim 1, wherein said subject is at risk of developing an allergic reaction to an agent suitable for increasing stability of a vaccine active compound or any vaccine excipient, and optionally wherein any one of: (i) the method further comprises further comprising a step preceding said administering, comprising determining said subject is at risk of developing an allergic reaction to any one of: an agent suitable for increasing stability of a vaccine active compound, and any vaccine excipient; (ii) said vaccine is a messenger RNA (mRNA)-based vaccine; (iii) said agent is Polyethylene glycol (PEG); (iv) said subject is at risk of developing an allergic reaction to PEG; and (v) any combination of (i) to (iv).

8-11. (canceled)

12. The method of claim 1, wherein said immunogenic polypeptide is derived from a viral peptide.

13. The method of claim 1, wherein said pathogen is a pathogenic virus, optionally wherein said pathogenic virus comprises a Coronavirus, and optionally wherein said Coronavirus comprises any one of: the Wuhan human Corona 2020 (SARS-COV2), SARS-COV, or MERS-COV, or any variant thereof.

14-15. (canceled)

16. The method of claim 1, wherein said subject has been afflicted with COVID-19, had been vaccinated against COVID-19, or both, and optionally wherein the subject had previously been vaccinated by an intramuscular injection or a subcutaneous injection of a composition comprising a SARS-COV2 spike protein and is orally administered with a therapeutically effective amount of a pharmaceutical composition comprising a SARS-COV2 spike protein receptor binding domain as an immunogenic polypeptide.

17. The method of claim 1, wherein said subject is characterized by having above a predetermined threshold of immunoglobulins having specific binding affinity to at least one peptide being derived from SARS-COV-2.

18. The method of claim 1, wherein said increasing immunization efficiency comprises inducing production, increasing levels, or both, of systemic neutralizing antibodies, in said subject.

19. The method of claim 1, wherein said increasing immunization efficiency comprises increasing any one of: the serum titer of IgG, mucosal IgA, mucosal antibody response, T cell immune response, or any combination thereof, in said subject, and optionally wherein said T cell comprises any one of a cytotoxic T cell and a T helper cell.

20. (canceled)

21. The method of claim 1, wherein said functional analog is characterized by having at least 80% sequence identity to said LTB.

22. The method of claim 1, wherein said LTB polypeptide comprises a plurality of LTB polypeptides.

23. The method of claim 1, wherein said immunogenic polypeptide comprises a plurality of immunogenic polypeptides comprising at least two viral peptides or any analogs thereof having at least 80% sequence identity to said at least two viral peptides, and optionally wherein any one of: (i) said plurality of immunogenic polypeptides comprises at least two viral peptides or any analogs thereof having at least 80% sequence identity to said at least two viral peptides; (ii) said pharmaceutical composition comprises a first viral peptide, and said LTB conjugated to at least a second viral peptide, thereby forming a chimeric polypeptide; and (iii) both (i) and (ii).

24-25. (canceled)

26. The method of claim 22, wherein any one of: (a) said plurality of LTB polypeptides comprises: (i) at least a first LTB polypeptide being a non-conjugated LTB; (ii) at least a second LTB polypeptide conjugated to at least one peptide of said plurality of immunogenic polypeptides, thereby forming a chimeric polypeptide; or (iii) any combination of (i) and (ii); (b) said plurality of LTB polypeptides comprises at least a first LTB polypeptide being a non-conjugated LTB and at least a second LTB polypeptide conjugated to at least one peptide of said plurality of immunogenic polypeptides, thereby forming a chimeric polypeptide; (c) said at least two viral peptides comprise (i) a viral spike protein; and (ii) a viral nucleocapsid protein, wherein said at least two viral peptides comprise the full length amino acid sequence or a partial amino acid sequence of said viral spike protein and of said viral nucleocapsid protein, or an analog of any one of said spike protein and of said nucleocapsid protein, having at least 80% sequence identity to any one of said spike protein and said nucleocapsid protein; and (d) any one of (a) to (c).

27-28. (canceled)

29. The method of claim 26, wherein any one of: (i) said spike protein comprises the amino acid sequence: ITNLCPFGEVENATRFASVYAWNRKRISNCVADYSVLYNSASFSTFKCYGVSPTKLNDL CFTNVYADSFVIRGDEVRQIAPGQTGKIADYNYKLPDDFTGCVIAWNSNNLDSKVGGN YNYLYRLFRKSNLKPFERDISTEIYQAGSTPCNGVEGENCYFPLQSYGFQPTNGVGYQPY RVVVLSFELLHAPATVCGPKKSTNL (SEQ ID NO: 15); or any analog thereof having at least 80% sequence identity thereto; (ii) said spike protein comprises the amino acid sequence: VNLTTRTQLPPAYTNSFTRGVYYPDKVFRSSVLHSTQDLFLPFFSNVTWFHAIHVSGTNG TKRFDNPVLPFNDGVYFASTEKSNIIRGWIFGTTLDSKTQSLLIVNNATNVVIKVCEFQFC NDPFLGVYYHKNNKSWMESEFR VYSSANNCTFEYVSQPFLMDLEGKQGNFKNLREFVF KNIDGYFKIYSKHTPINL VRDLPQGFSALEPLVDLPIGINITRFQTLLALHRSYLTPGDSSS GWTAGAAAYYVGYLQPRTFLLKYNENGTITDAVDCALDPLSETKCTLKSFTVEKGIYQ TSNFR VQPTESIVRFPNITNLCPFGEVENATRFASVYAWNRKRISNCVADYSVLYNSASF STFKCYGVSPTKLNDLCFTNVYADSFVIRGDEVRQIAPGQTGKIADYNYKLPDDFTGCVI AWNSNNLDSKVGGNYNYLYRLFRKSNLKPFERDISTEIYQAGSTPCNGVEGENCYFPLQ SYGFQPTNGVGYQPYRVVVLSFELLHAPATVCGPKKSTNLVKNKCVNFNFNGLTGTGV LTESNKKFLPFQQFGRDIADTTDAVRDPQTLEILDITPCSFGGVSVITPGTNTSNOVAVLY QDVNCTEVPVAIHADQLTPTWRVYSTGSNVFQTRAGCLIGAEHVNNSYECDIPIGAGIC ASYQTQTNSPRRAR (SEQ ID NO: 2); or any analog thereof having at least 80% sequence identity thereto; (iii) said nucleocapsid protein comprises the amino acid sequence: (a) NNTASWFTALTQHGKEDLKFPRGQGVPINTNSSPDDQIGYYRRATRRIRGGDGKMKDL SPRWYFYYLGTGPEAGLPYGANKDGIIWVATEGALNTPKDHIGTRNPANNAAIVLQLPQ GTTLPKGFYAEGS (SEQ ID NO: 3); (b) AAEASKKPRQKRTATKAYNVTQAFGRRGPEQTQGNFGDQELIRQGTDYKHWPQIAQFA PSASAFFGMSRIGMEVTPSGTWLTYTGAIKLDDKDPNFKDQVILLNKHIDAYKT (SEQ ID NO: 4); (c) MSDNGPQNORNAPRITFGGPSDSTGSNQNGERSGARSKQRRPQGLPNNTASWFTALTQ HGKEDLKFPRGQGVPINTNSSPDDQIGYYRRATRRIRGGDGKMKDLSPRWYFYYLGTG PEAGLPYGANKDGIIWVATEGALNTPKDHIGTRNPANNAAIVLQLPQGTTLPKGFYAEG SRGGSQASSRSSSRSRNSSRNSTPGSSRGTSPARMAGNGGDAALALLLLDRLNQLESKM SGKGQQQQGQTVTKKSAAEASKKPROKRTATKAYNVTQAFGRRGPEQTQGNFGDQEL IRQGTDYKHWPQIAQFAPSASAFFGMSRIGMEVTPSGTWLTYTGAIKLDDKDPNFKDQV ILLNKHIDAYKTFPPTEPKKDKKKKADETQALPQROKKQQTVTLLPAADLDDFSKQLQQ SMSSADSTQA (SEQ ID NO: 10); or (d) any analog having at least 80% sequence identity to SEQ ID NO: 3, SEQ ID NO: 4, or SEQ ID NO: 10; (iv) said nucleocapsid protein comprises the amino acid sequence: (a) NNTASWFTALTQHGKEDLKFPRGQGVPINTNSSPDDQIGYYRRATRRIRGGDGKMKDL SPRWYFYYLGTGPEAGLPYGANKDGIIWVATEGALNTPKDHIGTRNPANNAAIVLQLPQ GTTLPKGFYAEGS (SEQ ID NO: 3); (b) AAEASKKPROKRTATKAYNVTQAFGRRGPEQTQGNFGDQELIRQGTDYKHWPQIAQFA PSASAFFGMSRIGMEVTPSGTWLTYTGAIKLDDKDPNFKDQVILLNKHIDAYKT (SEQ ID NO: 4); (c) MSDNGPQNORNAPRITFGGPSDSTGSNONGERSGARSKQRRPQGLPNNTASWFTALTQ HGKEDLKFPRGQGVPINTNSSPDDQIGYYRRATRRIRGGDGKMKDLSPRWYFYYLGTG PEAGLPYGANKDGIIWVATEGALNTPKDHIGTRNPANNAAIVLOLPQGTTLPKGFYAEG SRGGSQASSRSSSRSRNSSRNSTPGSSRGTSPARMAGNGGDAALALLLLDRLNQLESKM SGKGQQQQGQTVTKKSAAEASKKPRQKRTATKAYNVTQAFGRRGPEQTQGNFGDQEL IRQGTDYKHWPQIAQFAPSASAFFGMSRIGMEVTPSGTWLTYTGAIKLDDKDPNFKDQV ILLNKHIDAYKTFPPTEPKKDKKKKADETQALPQRQKKQQTVTLLPAADLDDESKQLQQ SMSSADSTQA (SEQ ID NO: 10); or (d) any analog having at least 80% sequence identity to SEQ ID NO: 3, SEQ ID NO: 4, or SEQ ID NO: 10; (v) said conjugated is via a peptide linker comprising an amino acid sequence of 2 to 10 amino acids, optionally wherein said linker comprises 3 to 7 amino acids, said linker comprises Serine and Glycine amino acid residues, said linker consists of Serine and Glycine amino acid residues, or any combination thereof; (vi) said pharmaceutical composition comprises: (a) an LTB polypeptide being a non-conjugated LTB; and said at least two viral peptides; (b) a first LTB polypeptide being a non-conjugated LTB; a first viral peptide; and a chimeric polypeptide comprising at least a second LTB polypeptide conjugated to at least a second viral peptide; (c) a first chimeric polypeptide comprising a first LTB polypeptide conjugated to at least a first viral peptide and a second chimeric polypeptide comprising at least a second LTB polypeptide conjugated to at least a second viral peptide; (d) a first viral peptide; and a chimeric polypeptide comprising an LTB polypeptide conjugated to at least a second viral peptide; or (e) any combination of (a) to (d); and (vii) any combination of (i) to (vi).

30-36. (canceled)

37. The method of claim 26, wherein said chimeric polypeptide comprises the sequence of any one of:
(i)
(SEQ ID NO: 9)
ISMKNEFITNLCPFGEVENATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGENCYFPLQSYGFQPTNG
VGYQPYRVVVLSFELLHAPATVCGPKKSTNLVKNKXVNFNFNGLTGT,
wherein X is cysteine or alanine;
(ii)
(SEQ ID NO: 5)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGVNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDPLSETKCTLK
SFTVEKGIYQTSNFRVQPTESIVRFPNITNLCPFGEVENATRFASVYAWNRKRISNCVAD
YSVLYNSASFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQAGSTPCNG
VEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLIGAEHVNNS
YECDIPIGAGICASYQTQTNSPRRAR;
(iii)
(SEQ ID NO: 6)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGNNTASWFTALTQHGKEDLKFPRGQGVPINTNSSPDDQIGYYRRATRRIR
GGDGKMKDLSPRWYFYYLGTGPEAGLPYGANKDGIIWVATEGALNTPKDHIGTRNPAN
NAAIVLQLPQGTTLPKGFYAEGS;
(iv)
(SEQ ID NO: 7)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGAAEASKKPRQKRTATKAYNVTQAFGRRGPEQTQGNFGDQELIRQGTDY
KHWPQIAQFAPSASAFFGMSRIGMEVTPSGTWLTYTGAIKLDDKDPNFKDQVILLNKHI
DAYKT;
and
(v)
(SEQ ID NO: 11)
MGNKVKCYVLFTALLSSLYAHGAPQTITELCSEYRNTQIYTINDKILSYTESMAGKREM
VIITFKSGETFQVEVPGSQHIDSQKKAIERMKDTLRITYLTETKIDKLCVWNNKTPNSIAA
ISMKNGGSGGSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGKMKDLSPRW
YFYYLGTGPEAGLPYGANKDGIIWVATEGALNTPKDHIGTRNPANNAAIVLQLPQGTTL
PKGFYAEGSRGGSQASSRSSSRSRNSSRNSTPGSSRGTSPARMAGNGGDAALALLLLDR
LNQLESKMSGKGQQQQGQTVTKKSAAEASKKPROKRTATKAYNVTQAFGRRGPEQTQ
GNFGDQELIRQGTDYKHWPQIAQFAPSASAFFGMSRIGMEVTPSGTWLTYTGAIKLDDK
DPNFKDQVILLNKHIDAYKTFPPTEPKKDKKKKADETQALPQRQKKQQTVTLLPAADL
DDFSKQLQQSMSSADSTQA.

38-41. (canceled)

42. The method of claim 1, wherein said increasing is compared to a control subject.

43. (canceled)


SEQUENCE LISTING (18 sequences)

SEQ ID NO: 1 - protein, 125 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKN

SEQ ID NO: 2 - protein, 670 aa, Artificial Sequence
  Synthetic
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 130 aa, Artificial Sequence
  Synthetic
NNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGS

SEQ ID NO: 4 - protein, 112 aa, Artificial Sequence
  Synthetic
AAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKT

SEQ ID NO: 5 - protein, 800 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 6 - protein, 260 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGS

SEQ ID NO: 7 - protein, 242 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKT

SEQ ID NO: 8 - protein, 220 aa, Artificial Sequence
  Synthetic
  MISC_FEATURE (209)..(209): X is Cys or Ala
EFITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKXVNFNFNGLTGT

SEQ ID NO: 9 - protein, 350 aa, Artificial Sequence
  Synthetic
  MISC_FEATURE (339)..(339): X is Cys or Ala
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGEFITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKXVNFNFNGLTGT

SEQ ID NO: 10 - protein, 419 aa, Artificial Sequence
  Synthetic
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 548 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNGGSGGSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 12 - protein, 202 aa, Artificial Sequence
  Synthetic
ITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNL

SEQ ID NO: 13 - protein, 202 aa, Artificial Sequence
  Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNL

SEQ ID NO: 14 - protein, 202 aa, Artificial Sequence
  Synthetic
ITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNL

SEQ ID NO: 15 - protein, 202 aa, Artificial Sequence
  Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 16 - protein, 329 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNEFITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 17 - protein, 327 aa, Artificial Sequence
  Synthetic
MGNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 18 - protein, 567 aa, Artificial Sequence
  Synthetic
MKTIIALSYIFCLVFAQDLPGNDNNSTATLCLGHHAVPNGTLVKTITNDQIEVTNATELVQSSSTGKICNNPHRILDGINCTLIDALLGDPHCDGFQNEKWDLFVERSKAFSNCYPYDVPDYASLRSLVASSGTLEFINEGFNWTGVTQNGGSSACKRGPDSGFFSRLNWLYKSGSTYPVQNVTMPNNDNSDKLYIWGVHHPSTDKEQTNLYVQASGKVTVSTKRSQQTIIPNVGSRPWVRGLSSRISIYWTIVKPGDILVINSNGNLIAPRGYFKMRTGKSSIMRSDAPIGTCSSECITPNGSIPNDKPFQNVNKITYGACPKYVKQNTLKLATGMRNVPEKQTRGIFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI